SEQUENCE LISTING (8 sequences)

SEQ ID NO: 1 - RNA, 19 nt, Homo sapiens
ccaagaagaccguggacaa

SEQ ID NO: 2 - RNA, 19 nt, Homo sapiens
gcaagaagccggccgagua

SEQ ID NO: 3 - RNA, 19 nt, Homo sapiens
aauccauggaaguggccaa

SEQ ID NO: 4 - RNA, 19 nt, Homo sapiens
ccgcggacugcaagcggaa

SEQ ID NO: 5 - DNA, 20 bp, Artificial Sequence
  PRIMER
gagctgacggcggagaacga

SEQ ID NO: 6 - DNA, 20 bp, Artificial Sequence
  PRIMER
accccgcaggaacatcttta

SEQ ID NO: 7 - DNA, 21 bp, Artificial Sequence
  PRIMER
gaagtggccaacttctactac

SEQ ID NO: 8 - DNA, 20 bp, Artificial Sequence
  PRIMER
cgcctggtagccgaggtaag

(19) United States
(12) Patent Application Publication
(10) Pub. No.: US 2009/0263354 A1
(43) Pub. Date: Oct. 22, 2009

(54) Compositions and siRNAs for inhibiting C/EBPbeta

(75) Inventors: Menachem Rubinstein, Rehovot (IL); Ofir Meir, Rehovot (IL)

Correspondence Address: BROWDY AND NEIMARK, P.L.L.C.;624 NINTH STREET, NW, SUITE 300, WASHINGTON, DC, 20001-5303, US

(73) Assignee: Yeda Research and Development Co., Ltd., Rehovot (IL)

(21) Appl. No.: 12/382,182

(22) Filed: Mar. 10, 2009

Related U.S. Application Data
(63) Continuation-in-part of application No. PCT/IL2007/001112, filed on Sep. 10, 2007.

(30) Foreign Application Priority Data
Sep. 10, 2006 (IL) 177989

(51) Int. Cl. A61K 38/21; C07H 21/02; A61K 31/7088; A61K 38/19; A61P 35/00

(52) U.S. Cl. 424/85.5; 536/24.5; 514/44 A; 424/85.1

(57) ABSTRACT

The invention relates to C/EBPβ and modulation of cell resistance or sensitivity to triggers of cell death. More particular, it provides pharmaceutical compositions and siRNAs for inhibiting C/EBPβ thus decreasing resistance or enhancing sensitivity of cancer cells to a cancer therapy.

### CROSS-REFERENCE TO RELATED APPLICATIONS

The present application is a continuation-in-part application of PCT application No. PCT/IL2007/001112, filed Sep. 10, 2007, in which the US is designated, and claims the benefit of Israeli Patent Application No. 177989, filed Sep. 10, 2006, the entire contents of each and all these applications being hereby incorporated by reference herein in their entirety as if fully disclosed herein.

### FIELD OF THE INVENTION

The present invention relates to C/EBPβ and modulation of cell resistance or sensitivity to triggers of cell death. More particular, it relates to compositions and siRNAs for inhibiting C/EBPβ thus decreasing resistance or enhancing sensitivity of cancer cells to a cancer therapy.

### BACKGROUND OF THE INVENTION

The CCAAT/enhancer-binding protein family comprises six (C/EBPβ to ζ) basic leucine zipper transcription factors that are regarded as master regulators of cellular proliferation and differentiation, inflammation, and various other functions in multiple tissues (Ramji and Foka, 2002). C/EBPβ is an important transcriptional regulator of adipocyte, hepatocyte and macrophage differentiation, macrophage function and inflammatory response. The control of C/EBPβ expression and activity is complex and involves regulation at the transcriptional, translational and post-translational levels by stage- and tissue-specific mechanisms. Its functional diversity is achieved through an intrinsic ability to form heterodimers with many other transcription factors, including NF-κB p50 subunit, C/ATF, Fos and Jun, p300, IRF-1, CHOP-10, Smad3, cyclin D1 and p53 (Choy and Derynck, 2003; Lamb et al., 2003; Schneider-Merck et al., 2006).

The C/EBPβ gene is located on the long arm of chromosome 20 (20q), where DNA copy number amplification has been observed in a wide variety of cancers, including gastric, prostate, ovarian, colorectal, pancreatic cancers and glyoma (Vegesna et al., 2002; Sankpal et al., 2005; Homma et al., 2006). C/EBPβ is a survival factor in Wilms tumor cells and is essential for myc/raf-induced transformation of macrophages (Wessells et al., 2004; Li et al., 2005). Importantly, it was implicated as a critical effector of cyclin D1 action, contributing materially to the development of human cancer (Lamb et al., 2003).

Very few publications link C/EBPβ with apoptosis and no clear trend have emerged. Studies carried out with neuronal cells (Cortes-Canteli et al., 2002) show that in these cells C/EBPβ is involved in pathways leading to apoptosis through activation of p53. The publication examines the effect of C/EBPβ over-expression on differentiation and apoptosis of mouse neuronal cells induced by serum withdrawal. Increased differentiation and apoptosis in C/EBPβ over-expressing cells was observed, and it was suggested that apoptosis was induced by C/EBPβ over-expression via activation of p53 protein and cdk inhibitor p21.

Additional studies carried out with neuronal cells (Marshall et al., 2003) showed that inhibiting C/EBPβ activity with a dominant-negative C/EBP mutant enhances neuronal survival in the absence of IGF-1, that specific inhibition of C/EBPβ with specific siRNA or antisense improves survival and that both specific siRNA or antisense approaches antagonized NMDA-mediated death. In contrast, studies employing bone marrow cells (Wessells et al., 2004) show that C/EBPβ has pro-oncogenic effects and that the IGF-I gene is a candidate to mediate the pro-oncogenic effect of C/EBPβ. The results presented show that C/EBPβ^{-/-} (knock out) bone marrow cells were refractory to transformation induced by a carcinogen derived from retrovirus and that such cells were dependent on exogenous growth factor for proliferation and/or survival. Wessels et al. observed that C/EBPβ deficiency does not affect apoptosis caused by M-CSF withdrawal of normal non-transformed bone marrow cells.

One group working with stellated cells (Buck et al., 2001) showed that deficiency of C/EBPβ activity does affect apoptosis induced by exposure of the cells to CCL4 in non transformed stellated cells. It was observed that the ability of C/EBPβ to prevent CCL4 induced apoptosis in stellated cells required phosphorylation of Thr217 by the p90 Rsk kinase, and in generating an XEXD-like motif that serves as a caspase inhibitor site.

In the model of Wessels et al. with bone marrow cells the C/EBPβ T271A mutant was nearly as effective as wt C/EBPβ in restoring the colony formation of transformed C/EBPβ^{-/-} bone marrow cells indicating that phosphorylation of Thr217 by Rsk is clearly dispensable for the ability of C/EBPβ to inhibit apoptosis in transformed bone marrow cells.

Wessels et al. showed also that C/EBPβ^{-/-} tumor derived macrophage cells displayed increased resistance to apoptosis induced by withdrawal of M-CSF, due to increased IGF-I expression in these cells.

Schneider-Merck et al. (2006) disclosed the reciprocal inhibition between C/EBPβ, and the tumor suppressor protein p53. They observed repression of p53 transcriptional activity by C/EBPβ. The physical interaction of p53 and C/EBPβ was verified by co-immunoprecipitation. The authors indicate that deregulation of C/EBPβ and p53 crosstalk is implicated in tumorigenesis and that enhanced expression of C/EBPβ could antagonize the tumor-suppressive role of p53.

### SUMMARY OF THE INVENTION

In one aspect, the present invention relates to a C/EBPβ, specific siRNA selected from the C/EBPβ specific siRNAs of SEQ ID NOs: 1-4 or chemically modified derivatives thereof.

| | |
|---|---|
| SEQ ID NO: 1 | 5′ CCAAGAAGACCGUGGACAA |
| SEQ ID NO: 2 | 5′ GCAAGAAGCCGGCCGAGUA |
| SEQ ID NO: 3 | 5′ AAUCCAUGGAAGUGGCCAA |
| SEQ ID NO: 4 | 5′ CCGCGGACUGCAAGCGGAA |

In one embodiment, the chemically modified C/EBPβ specific siRNA of the invention is chol-C/EBPβ, specific siRNA, i.e., a C/EBPβ, specific siRNA linked to cholesterol.

In another aspect, the present invention provides a pharmaceutical composition comprising a C/EBPβ specific siRNA as defined above and a pharmaceutically acceptable carrier. This composition may be used for decreasing resistance or enhancing sensitivity of cancer cells to a cancer therapy, preferably for decreasing resistance of cancer cells to a cancer therapy.

In still another aspect, the present invention provides a pharmaceutical composition comprising CHOP-10, a CHOP-10 expression vector or an inducer of CHOP-10, and a pharmaceutically acceptable carrier, for decreasing resistance or enhancing sensitivity of cancer cells to a cancer therapy.

In one embodiment of the aforesaid compositions, the resistance of the cancer cells to the cancer therapy is not associated to p53 multidrug resistance gene 1 (MDR1), IGF-1, IL-6 or AKT activity and/or level.

In certain embodiments of these compositions, the cancer therapy induces apoptosis in cells. In other embodiments, said compositions are useful for decreasing resistance or enhancing sensitivity of cancer cells to a cancer therapy such as chemotherapy, cytokine therapy, preferably comprising administration of FasL, IFN-γ, TNF-α or a combination thereof, proteasome inhibitor therapy, preferably comprising administration of MG262, lactacystin, ALLN or a combination thereof, radiation therapy, or a combination thereof.

In a further aspect, the present invention relates to the use of an inhibitor of C/EBPβ in the manufacture of a pharmaceutical composition for decreasing resistance or enhancing sensitivity of cancer cells to a cancer therapy.

In one embodiment, the invention relates to decreasing resistance of cancer cells to a cancer therapy.

In another embodiment, the invention relates to cancer cells in which their resistance to the cancer therapy is not associated to p53 multidrug resistance gene 1 (MDR1), IGF-1, IL-6 or AKT activity and/or level.

In certain embodiments, the invention relates to a cancer therapy that induces apoptosis in cells.

In a further embodiment, the invention relates to a cancer therapy such as chemotherapy, cytokine therapy, proteasome inhibitor therapy, radiation therapy or a combination thereof.

In certain embodiments, the invention relates to a proteasome inhibitor therapy, which comprises administration of MG262, lactacystin, ALLN or a combination thereof, or to a cytokine therapy, which comprises administration of FasL, IFN-γ, TNF-α or a combination thereof.

In a further embodiment, the invention relates to a C/EBPβ inhibitor including, but not limited to, (i) C/EBPβ specific siRNA or shRNA; (ii) CHOP-10, a CHOP-10 expression vector and/or inducers of CHOP-10; (iii) an inhibitory small molecule of C/EBPβ identified in a screening assay; (iv) a dominant negative mutant of C/EBPβ; and/or (v) C/EBPβ specific antibodies.

In one embodiment, the C/EBPβ inhibitor is a C/EBPβ specific siRNA, preferably selected from the C/EBPβ specific siRNAs of SEQ ID NOs: 1-4.

In certain embodiments, the C/EBPβ inhibitor is a C/EBPβ specific siRNA chemically modified to increase its penetration into cancer cells, such as chol-C/EBPβ specific siRNA; a CHOP-10 expression vector; and/or a small molecule identified in a screening assay.

In a further embodiment, the screening assay comprises measuring expression of a reporter gene in cells harbouring a reporter gene controlled by a promoter responsive to C/EBPβ, with or without transfection with a recombinant C/EBPβ expression vector, in the presence or absence of a small molecule.

In another embodiment, the C/EBPβ inhibitor is linked to a ligand, which binds specifically to cancer cells.

In still a further aspect, the present invention relates to the use of at least one of the following agents: (i) C/EBPβ or a mutein, isoform, fused protein, functional derivative, active fraction, circularly permutated derivative or a salt thereof; (ii) an expression vector encoding (i); and/or (iii) an activating small molecule of C/EBPβ identified in a screening assay in the manufacture of a medicament for enhancing resistance or decreasing sensitivity of normal cells to a cancer therapy, thereby preventing or treating side effects of cancer therapy.

In one embodiment, the invention relates to a cancer therapy such as chemotherapy, cytokine therapy, proteasome inhibitor therapy, radiation therapy or a combination thereof.

In a further embodiment, the proteasome inhibitor therapy comprises administration of MG262, lactacystin, ALLN or a combination thereof; the cytokine therapy comprises administration of FasL, IFN-γ, TNF-α or a combination thereof; and the chemotherapy comprises administration of doxorubicin.

In yet a further embodiment, the invention relates to an agent that is a C/EBPβ expression vector having a cardiac specific promoter placed upstream of the C/EBPβ gene in the C/EBPβ expression vector.

In still another embodiment, the invention relates to an agent that is a C/EBPβ activating small molecule identified in a screening assay. The C/EBPβ activating small molecule may be obtainable by using an assay comprising measuring expression of a reporter gene in cells harboring a reporter gene controlled by a promoter responsive to C/EBPβ and a recombinant C/EBPβ gene in the presence or absence of a small molecule.

In certain embodiments, the cancer therapy induces apoptosis in cells.

In yet a further aspect, the present invention relates to the use of a C/EBPβexpression vector allowing C/EBPβ expression in normal cells but not in a cancer cell in combination with a C/EBPβ inhibitor targeted to the cancer cell in the manufacture of a medicament for decreasing resistance of a cancer cells to a cancer therapy and increasing resistance of normal cells to the cancer therapy, thereby preventing or treating side effects of the cancer therapy.

In one embodiment, the resistance of the cancer cells to said cancer therapy is not associated to p53 multidrug resistance gene 1 (MDR1), IGF-1, IL-6 or AKT activity and/or level.

In another embodiment, targeting to a cancer cell is achieved by linking the C/EBPβ inhibitor to a ligand, which binds specifically to the cancer cell.

In a further embodiment, the C/EBPβ inhibitor is C/EBPβ specific siRNA chemically modified to increase its penetration into cancer cells such as chol-C/EBP specific siRNA.

In another embodiment, the C/EBPβ inhibitor is a CHOP-10 expression vector.

In still another embodiment, the C/EBPβ inhibitor is a small molecule identified in a screening assay.

In a further embodiment, the cancer therapy is chemotherapy, cytokine therapy, proteasome inhibitor therapy, radiation therapy or a combination thereof.

In certain embodiments, the proteasome inhibitor therapy comprises administration of MG262, lactacystin, ALLN or a combination thereof; the cytokine therapy comprises administration of FasL, IFN-γ, TNF-α or a combination thereof; and the chemotherapy comprises administration of doxorubicin.

In a further embodiment, the cancer therapy comprises administration of doxorubicin and a cardiac specific promoter is placed upstream of the C/EBPβ gene in the C/EBPβ expression vector.

In still another aspect, the present invention provides a method for decreasing resistance or enhancing sensitivity of cancer cells to a cancer therapy in an individual in need, comprising administering to said individual an effective amount of a C/EBPβ inhibitor selected from (i) a C/EBPβ specific siRNA or shRNA; or (ii) CHOP-10, a CHOP-10 expression vector and/or an inducer of CHOP-10.

In one embodiment, the C/EBPβ inhibitor is a C/EBPβ specific siRNA, preferably selected from the C/EBPβ specific siRNAs of SEQ ID NOs: 1-4.

In another embodiment, the C/EBPβ specific siRNA is chemically modified to increase its penetration into cancer cells. The chemically modified C/EBPβ specific siRNA may be, e.g., chol-C/EBPβ specific siRNA.

In a further embodiment, the C/EBPβ inhibitor is a CHOP-10 expression vector.

In yet another embodiment, the C/EBPβ inhibitor is linked to a ligand, which binds specifically to cancer cells.

In certain embodiments, the cancer therapy induces apoptosis in cells. In other embodiments, said method is useful for decreasing resistance or enhancing sensitivity of cancer cells to a cancer therapy such as chemotherapy, cytokine therapy, preferably comprising administration of FasL, IFN-γ, TNF-α or a combination thereof, proteasome inhibitor therapy, preferably comprising administration of MG262, lactacystin, ALLN or a combination thereof, radiation therapy, or a combination thereof.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1C show enhanced resistance of human WISH cells over-expressing C/EBPβ to induction of death by the non-specific proteasome inhibitor ALLN. Cells were transfected either with empty vector (pCDNA3, Promega) or with C/EBPβ expression vector (pC-C/EBPβ), grown in 6-well plates, treated with medium alone (Control) or with ALLN in medium. The cultures were then observed under the microscope (A), then stained with crystal violet and photographed (B). Alternatively, cell viability was evaluated at the indicated times after addition of ALLN treatment and following staining with a viability stain (C).

FIG. 2 shows enhanced resistance of human WISH cells over-expressing C/EBPβ to different inducers of cell death. Cells were transfected either with empty vector (pCDNA3, Promega) or with C/EBPβ expression vector (pC-C/EBPβ), grown in 6-well plates, treated with medium alone (Control) or with the indicated inducers of cell death. The cultures were then stained with crystal violet and photographed (left photos) and then observed under the microscope (magnification ×100, right photos).

FIG. 3 shows enhanced resistance of human WISH cells over-expressing C/EBPβ to inducers of cell death. Cells were transfected either with empty vector (pCDNA3, Promega) or with C/EBPβ expression vector pC-C/EBPβ, grown in 6-well plates, treated the indicated inducers of cell death. The cultures were then stained with crystal violet and photographed (left photos) and then observed under the microscope (magnification ×100, right photos).

FIG. 4 shows apoptosis of human WISH cells treated with the proteosome inhibitor MG262 by fluorescence analysis cell sorting (FACS) and protection of cells from MG262 induced apoptosis by C/EBPβ. Cells were transfected either with control vector (pCDNA3, Promega) or with C/EBPβ expression vector pC-C/EBPβ, grown in 6-well plates, treated with the proteasome inhibitor MG262 and stained with anti-annexin V and propidium iodide. The cultures were then analyzed by FACS to distinguish between intact or apoptotic cells and between early and late apoptosis.

FIG. 5 shows the resistance of several human cell types to various inducers of cell death upon over-expression of C/EBPβ. Cells were transfected either with control vector (pCDNA3, Promega) or with C/EBPβ expression vector pC-C/EBPβ, grown in 6-well plates, treated with the indicated inducers of cell death. The cultures were then stained with crystal violet and photographed (left photos) and then observed microscopically (right photos).

FIG. 6 shows the inhibition of C/EBPβ-induced resistance to cell death by over-expression of the C/EBPβ antagonist CHOP-10. WISH cells were transfected either with control vector (pCDNA3), with a combination of C/EBPβ expression vector pC-C/EBPβ plus pCDNA3, or with a combination of pC-C/EBPβ plus CHOP-10 expression vector pC-CHOP-10. Total DNA concentration was kept the same in all cases. The cells were grown in 6-well plates and treated with TNFα plus IFNγ for 24 h. The cultures were then stained with crystal violet and photographed.

FIGS. 7A-7B show murine B16 melanoma cells stably transfected with an inducible C/EBPβ vector (A) reverse-transcription PCR of RNA isolated from murine B16 melanoma cells stably expressing an inducible C/EBPβ vector. Induction with doxycycline led to profound expression of C/EBPβ mRNA (A), as well as C/EBPβ protein, as determined by immunoblotting (B).

FIG. 8 shows the effect of C/EBPβ induction in B16 melanoma cells on their resistance to cell death induced by doxorubicin. Cells were grown in 6-well plates, C/EBPβ was induced by treatment with doxycycline. The cultures were then treated with medium alone (Control) or with doxorubicin. The cultures were then stained with crystal violet and photographed (left photos) and then observed microscopically (right photos).

FIG. 9 shows the effect of C/EBPβ induction in B16 melanoma cells on their resistance to cell death induced either by TNFα plus IFNγ or by ALLN. Cells were grown in 6-well plates, C/EBPβ was induced with doxycycline. The cultures were then treated with TNFα plus IFNγ or with doxorubicin. The cultures were then stained with crystal violet and photographed (left photos) and then observed microscopically (right photos).

FIG. 10 shows the effect of C/EBPβ induction in B16 melanoma cells on their resistance to cell death induced either by tunicamycin or by etoposide. Cells were grown in 6-well plates and C/EBPβ was induced with doxycycline. The cultures were then treated with tunicamycin or by etoposide. The cultures were then stained with crystal violet and photographed (left photos) and then observed microscopically (right photos).

FIGS. 11A-11B show ablation of C/EBPβ expression in WISH cells by siRNA (A) reverse-transcription PCR of RNA isolated from WISH cells transfected with C/EBPβ siRNA. Such transfection ablated the expression of C/EBPβ mRNA (A), as well as C/EBPβ protein, as determined by immunoblotting (B).

FIG. 12 shows the effect of C/EBPβ siRNA on death of WISH cells_induced by the proteasome inhibitor MG262. Cells were grown in 6-well plates, transfected with control siRNA or C/EBPβ siRNA and after 48 h treated with MG262. The cultures were then stained with crystal violet and photographed (left photos) and then observed microscopically (right photos).

FIG. 13 shows the effect of C/EBPβ siRNA on induction of apoptosis of WISH cells by the proteasome inhibitor MG262. Cells were grown in 6-well plates, transfected with control siRNA or C/EBPβ siRNA and after 48 h treated with MG262. The cultures were then stained with anti-annexin V and propidium iodide. The cultures were then analyzed by FACS to distinguish between intact or apoptotic cells and between early and late apoptosis.

FIG. 14 shows FACS of WISH cells transfected either with control siRNA (upper two graphs) or with C/EBPβ siRNA (lower two graphs). The cells were then either not treated (left two graphs) or treated with ALLN (26 μM) (right two graphs)

FIG. 15 shows FACS of WISH cells transfected either with control siRNA (upper two graphs) or with C/EBPβ siRNA (lower two graphs). The cells were then either not treated (left two graphs) or treated with TNFα plus IFNγ (right two graphs).

FIG. 16 shows FACS of human HeLa cells transfected either with control siRNA (left) or with C/EBPβ siRNA (right). The cells were then treated with TNFα plus IFNγ and stained with antibodies to Annexin V and with propidium iodide.

FIG. 17 shows FACS of human MCF7 cells transfected either with control siRNA (left) or with C/EBPβ siRNA (right). The cells were then treated with TNFα plus IFNγ and stained with antibodies to Annexin V and with propidium iodide.

FIG. 18 shows immunoblotting analysis with antibodies to p53 and to α-actin of extracts from WISH cells treated with the indicated inducers of cell death.

FIG. 19 shows inhibition of induction of a p53 luciferase reporter vector by overexpression of MDM2 and insignificant inhibition of the p53 luciferase reporter vector by over-expression of C/EBPβ in cells. Human WISH cells were transfected with the p53 reporter vector together with control pCDNA3 vector, pC-C/EBPβ expression vector or pCMDM2 expression vector. The cells were then treated with the p53 inducer doxorubicin and fold induction of luciferase activity was determined.

FIG. 20 shows the resistance of human WISH cells to various inducers of cell death. Cells were transfected either with pC-C/EBPβ or with pCMDM2 expression vector. The cells were then grown in 6-well plates, treated with the indicated inducers of cell death. The cultures were then stained with crystal violet and photographed.

FIG. 21 shows lack of effect of the MDR1 inhibitor verapamil on resistance to cell death induced by a C/EBPβ expression vector. Human WISH cells were transfected either with control vector pCDNA3 or with pC-C/EBPβ expression vector. The cells were then grown in 6-well plates, treated for 24 h with the cell death inducer MG262 and either medium or medium containing verapamil. The cultures were then stained with crystal violet and photographed.

FIG. 22 shows lack of effect of IGF-1on cell death induced by adriamycin (doxorubicin). Human WISH cells were treated with IGF-1 (200 ng/ml) for the indicated times in 6-well plates. The cells were then treated with the cell death inducer adriamycin (doxorubicin) (2 μM, 16 h). The cultures were then stained with crystal violet and photographed.

FIG. 23 shows lack of effect of IL-6 on cell death induced by ALLN. Human WISH cells in 96 well plated were treated with the indicated concentrations of IL-6. The cells were then treated either with medium or with the cell death inducer ALLN. The cultures were then stained with crystal violet and photographed.

FIG. 24 shows lack of effect of antibodies to the IL-6 receptor gp130 on C/EBPβ-induced protection from cell death induced by the combination of TNF-α and IFN-γ. Cells were transfected either with control vector (pCDNA3, Promega) or with C/EBPβ expression vector pC-C/EBPβ, grown in 6-well plates, treated with medium alone (Control) or with antibody to gp130. All cultures were then treated with the combination of TNFα (100 ng/ml) plus IFNγ (1000 IU/ml). The cultures were then stained with crystal violet and photographed.

FIG. 25 shows lack of effect of the AKT inhibitor LY294002 on C/EBPβ-induced protection from cell death mediated by TNFα plus IFNγ. Cells were transfected either with control vector (pCDNA3, Promega) or with pC-C/EBPβexpression vector, grown in 6-well plates, treated with medium alone (Control) or with medium containing the AKT inhibitor LY294002. All cultures were then treated with the combination of TNFα (100 ng/ml) plus IFNγ (1000 IU/ml). The cultures were then stained with crystal violet and photographed.

FIG. 26 shows images, shot by the iVIS digital camcorder, of doxycycline (+doxy) and control (−doxy) treated tumors, developed following subcutaneous injection of murine B16 melanoma Clone F10.9-3 cells (0.5×10⁶, 100 μl) together with matrigel (100 μl, BD) to C57B1/6 mice, wherein doxycycline (1 mg/ml) was added to the drinking water of the doxycycline group for 7 days.

FIGS. 27A-27C show a plot of the tumor weights (mg), developed following subcutaneous injection of murine B16 melanoma Clone F10.9-3 LAP cells or F10.9-4 LIP cells (0.5×10⁶, 100 μl) together with matrigel (100 μl, BD) to 4 groups of 10 C57B1/6 mice, wherein doxycycline (1 mg/ml) was added to the drinking water of the doxycycline groups for 13 days (27A); as well as photographs of typical tumors developed in each one of the groups (27B-27C—Clones F10.9-3 LAP and F10.9-4 LIP, respectively; +doxy/−doxy—with/without doxycycline).

### DETAILED DESCRIPTION OF THE INVENTION

According to the present invention it has been found that reduction of C/EBPβ activity in tumor cells can increase the response of these cells to a broad range of anti-cancer treatments, and that enhancement of C/EBPβ activity in normal cells may be useful to protect them from the deleterious effects of a broad range of anti-cancer treatments.

The invention is based on our findings showing that over-expression of C/EBPβ conferred resistance to a broad range of cell death inducers whereas knockdown of basal C/EBPβ by siRNA increased the sensitivity of various cells to inducers of cell death.

Thus, in one aspect, the invention relates to a C/EBPβ specific siRNA selected from the C/EBPβ specific siRNAs of SEQ ID NOs: 1-4 or chemically modified derivatives thereof.

In other aspects, the present invention provides a pharmaceutical composition comprising a C/EBPβ specific siRNA as defined above and a pharmaceutically acceptable carrier; as well as a pharmaceutical composition comprising CHOP-10, a CHOP-10 expression vector or an inducer of CHOP-10, and a pharmaceutically acceptable carrier, for decreasing resistance or enhancing sensitivity of cancer cells to a cancer therapy.

In a further aspect, the invention relates to the use of an inhibitor of C/EBPβ in the manufacture of a pharmaceutical composition for decreasing resistance or enhancing sensitivity of cancer cells to apoptosis induced by a cancer therapy. An inhibitor of C/EBPβ is capable of decreasing the levels or activity of C/EBPβ in cancer cells and is administered before, during or after cancer therapy administration to increase sensitivity or lower resistance of cancer cells to cancer therapy.

In still a further aspect, the present invention provides a method for decreasing resistance or enhancing sensitivity of cancer cells to a cancer therapy in an individual in need, comprising administering to said individual an effective amount of a C/EBPβ inhibitor selected from (i) a C/EBPβ specific siRNA or shRNA; or (ii) CHOP-10, a CHOP-10 expression vector and/or an inducer of CHOP-10.

We show herein for the first time that inhibiting C/EBPβ in cancer cells can enhance the sensitivity of the cancer cells to cancer-therapy. Enhancement of the sensitivity of cancer cells to cancer-therapy is crucial in patients harboring cancer cells, which are or become resistant to a wide variety of cancer drugs and therefore do not respond to cancer therapy.

We show herein for the first time that activating C/EBPβ in normal cells can protect these cells from the deleterious effects of cancer therapy. For example, it is of great importance to protect hematopoietic stem cells, which are very sensitive to cancer therapy. Thus activation of C/EBPβ in these cells may be used to protect them from cancer therapy. An activator of C/EBPβ is capable of increasing the levels or activity of C/EBPβ in normal cells and is administered before during or after cancer therapy administration to protect normal cells from cancer therapy.

The concept of increasing C/EBPβ for protection of normal cells from cancer therapy or decreasing of C/EBPβ for enhancement of cancer cell sensitivity to cancer therapy is new and could not be anticipated or predicted since the literature did not establish a clear role of C/EBPβ in apoptosis.

We found in accordance with the present invention that cells transfected with an expression plasmid encoding C/EBP (pC-C/EBPβ) acquired resistance to apoptosis induced by the non-specific proteasome inhibitor ALLN. Protection was evaluated by gross appearance of the cell culture in 6-well plates and by microscopy, as compared with cells transfected with the empty vector pCDNA3.

To evaluate the generality of the C/EBPβ-mediated protection, we repeated the experiments using a broad range of cell-death inducers, including the specific proteasome inhibitors MG262 and lactacystin, doxorubicin, γ-radiation, tunicamycin (an inducer of endoplasmic reticulum stress), a combination of TNFα and IFN-γ, and FAS ligand. In all of these cases, over-expression of C/EBPβ led to extensive protection from cell death.

Thus, we showed that over-expression of C/EBPβ conferred resistance to the following cell death inducers: doxorubicin, γ-radiation, proteasome inhibitors (MG262, lactacystin and ALLN), an inducer of endoplasmic reticulum (ER) stress (tunicamycin) and cytotoxic cytokines (TNFα and IFN-γ, FasL).

Chemotherapeutic agents and γ-irradiation induce apoptosis (also called programmed cell death) signaling, which converges in the activation of initiator caspases (e.g., procaspase-8, procaspase-9), resulting in the proteolytic activation of effector caspases (e.g., caspase-3) that cleave specific substrates.

To test for the mode of protection we performed 2D flow cytometry of the cell culture following transfection and treatment with the proteasome inhibitor MG262. Staining for annexin, a marker of apoptosis, and propidium iodide, a marker of membrane damage which serve to evaluate the progression of the apoptosis, we found that over-expression of C/EBPβ reduced both early and late apoptosis. Thus, we concluded that over-expression of C/EBPβ inhibits apoptosis induced by the specific proteasome inhibitor MG262.

We then tested different cell lines to further evaluate the generality of the protective effect exerted by over-expression of C/EBPβ. We found that such over-expression led to extensive protection of different cell types to different inducers of cell death. For example, over-expression of C/EBPβ protected HaCaT keratinocytes and HeLa cells (cervical carcinoma) from cell death induced by a combination of TNFα and IFN-γ. Over-expression of C/EBPβ protected MCF7 cells (breast carcinoma) from cell death induced by doxorubicin.

In order to show that the protective effect observed following transfection with C/EBPβ expression vector is indeed mediated by over-expression of C/EBPβ we employed CHOP-10 (also known as GADD153 or DDIT-3), a known inhibitor of C/EBPβ activity. Upon co-expression of C/EBPβ and CHOP-10 the protective effect of C/EBPβ was abolished.

To further test whether protection by over-expression of C/EBPβ was independent of the transfection process, we employed mouse melanoma B16 cells, to which an inducible C/EBPβ expression vector was stably introduced. Expression of C/EBPβ was induced in these cells by treatment with the antibiotic doxycycline, as demonstrated by RT-PCR and by immunoblotting with antibody directed against C/EBPβ. Upon induction of C/EBPβ expression, the cells became resistant to doxorubicin as demonstrated by staining of the culture and macroscopic and microscopic observation. Similar protection was observed upon challenge with ALLN, tunicamycin, etoposide, or a combination of TNF and IFN-γ.

To determine the role of endogenously expressed C/EBPβ in cell survival, we silenced it with siRNA, in particular, with a C/EBPβ siRNA pool comprising the siRNA of SEQ ID NOs: 1-4. Efficient silencing was obtained at 48 h post transfection, as determined by RT-PCR and by immunoblotting. Silencing of endogenous C/EBPβ increased significantly the sensitivity of WISH cells to apoptosis induced by MG262 as determined by macroscopic and microscopic observation and by annexin staining, as compared with cells transfected by control siRNA. The same effect of siRNA was observed upon challenge with either ALLN or a combination of TNF and IFN-γ. Thus, we showed that the knockdown of basal C/EBPβ by siRNA increased the sensitivity of cells to different inducers of cell death.

It was recently reported that C/EBPβ binds to p53 and inhibits its activity, part of which could be induction of apoptosis (Schneider-Merck et al., 2006). We performed several experiments to find out if the protective effect of C/EBPβ against apoptosis involves inhibition of p53. Initially, we determined which of the agents used for induction of apoptosis elevates the level of cellular p53. We found that etoposide, a combination of TNF and IFN-γ, vincristine, FasL and tunicamycin did not induce p53 in human WISH cells as determined by immunoblotting with antibody directed against p53, whereas extensive induction of p53 was obtained with MG262 and doxorubicin. WISH cells were then transfected with a p53 reporter vector together with pC-MDM2 expression vector (as a positive control for inhibition of p53), pC-C/EBPβ expression vector or pCDNA3 control vector. Following induction of p53 by doxorubicin, we found that pCMDM2 effectively inhibited p53 activity whereas only a slight inhibition was seen with pC-C/EBPβ. We then tested if MDM2 could mimic C/EBPβ in protecting from apoptosis triggered by p53 inducers. Both MG262 and doxorubicin induced massive apoptosis in cells transfected with pC-MDM2 whereas cells transfected with pC-C/EBPβ were protected. We therefore concluded that the protection from apoptosis elicited by over-expression of C/EBPβ is not mediated by inhibition of p53.

A C/EBPβ response element was identified in promoters of several other genes encoding pro-survival factors. We found that none of the pro-survival factors tested, multidrug resistance gene 1 (MDR1), IGF-1 (known to be induced by C/EBP-β, IL-6, and AKT was a mediator of the C/EBPβ-induced protective effect. We employed well-established methods to test for these mechanisms. Thus, verapamil, an inhibitor of MDR1, did not reverse the C/EBP-β-induced protective effect. Addition of IGF-1 to the culture medium did not protect WISH cells from doxorubicin or from MG262 or tunicamycin or a combination of TNF-α and IFN-γ. Another growth factor whose promoter contains a C/EBP response element is IL-6. Yet IL-6 added 6 h before challenge did not protect cells from apoptosis induced by ALLN. Similarly, antibodies to the IL-6 receptor signaling subunit gp130 that were added 2 h before challenge did not block the protective effect elicited by C/EBPβagainst the combination of TNF-α and IFN-γ in WISH cells. Finally, induction of AKT by C/EBPβ may lead to a signaling cascade involving inhibition of Bad and hence inhibition of apoptosis. However, the AKT inhibitor LY294002 did not reverse the protective effect of C/EBPβ.

These results indicate that modulating resistance or sensitivity of cells to apoptotic death by C/EBPβ is independent of p53, MDR1, IGF-1, IL-6 and AKT modulation and hence it represents a previously undisclosed mechanism that appears to be very general and suitable to a broad range of anti-cancer therapies.

C/EBPβ is expressed in two different forms, i.e., as an active form termed C/EBPβ LAP (LAP) and as a shorter inactive form termed C/EBPβ LIP (LIP), by using alternative translation start sites (Ramji and Foka, 2002), wherein LIP acts as a specific inhibitor of LAP (Descombes and Schibler, 1991). LAP is over-expressed in many types of tumors and was implicated in tumor cell growth and survival (Sebastian and Johnson, 2006; Lamb et al., 2003). A survey of primary cancer cells and cell lines identified a highly statistically significant link between cyclin D1 and C/EBPβ expression in many human cancers. Thus, together with cyclin D1, LAP accelerates cell proliferation (Lamb et al., 2003). Many other studies also implicated C/EBPβ in regulating cell death; however, a clear mechanism has not been established (Sterneck et al., 2006; Yoon et al., 2007).

To determine the role of C/EBPβ in tumor growth in mice, we used two different murine B16 melanoma clones, stably transfected with doxycycline-inducible LAP or LIP were used, and found that over-expression of LAP had a remarkable tumor growth-promoting effect, whereas over-expression of LIP inhibited tumor growth, probably by inhibiting basal expression of endogenous LAP.

The present invention discloses a positive correlation between C/EBPβexpression level in cells and resistance to cell death. Therefore, an inhibitor of C/EBPβ expression or activity may be useful in increasing the response of tumor cells to various cancer therapies, including but not limited to, chemotherapy, gamma-radiation, cytokines, proteasome inhibitors and inducers of endoplasmic reticulum stress. Specific agents that were tested include ALLN, MG262, lactacystin, a combination of TNFα and IFN-γ, FasL, doxorubicin, etoposide, tunicamycin and gamma radiation.

An example of an inhibitor of C/EBPβ expression is a C/EBPβ specific siRNA as shown in the examples below. The siRNA may comprise at least one of the sequences herein designated SEQ ID NOs: 1-4.

In recent years siRNA has been widely used for post-transcriptional silencing of specific mRNA targets (Dorsett and Tuschl, 2004). Target specificity in RNAi is achieved through RNA-RNA sequence recognition and base pairing. The siRNA consists of dsRNA, typically 19-21 bp long, with two nucleotides overhanging at each 3′ end. For maximal stability, two 2′ deoxynucleotides are used as 3′ overhangs. Alternatively, 27-mer blunt-ended nucleotides may be used, as these have shown improved efficiency in gene silencing (Kim et al., 2005). Transport of such siRNA molecules into cells may be enhanced by encapsulation into liposomes or by covalent coupling to highly lipophilic agents. Soutschek et al. (2004) showed intra venal administration of chemically modified siRNA specific to apoB linked to cholesterol (chol-apoB-siRNA) which was detected in the liver and jejunum of injected mice, significantly reduced the apoB mRNA levels in these organs, and reduced total cholesterol in the blood. Chol-siRNAs showed broad tissue bio-distribution 24 hours after injection of mice, and improved pharmacokinetic properties as compared to un-conjugated siRNAs.

Thus a chemically modified C/EBPβ specific siRNA, such as chol-C/EBPβspecific siRNA may be used for systemic administration to increase its penetration into tumor cells, thereby increasing the sensitivity of such tumor cells to cancer therapy. Alternatively, ligands, which bind specifically to cancer cells, may be linked to C/EBPβ inhibitory small molecules, such as siRNA, or vectors expressing inhibitors of C/EBPβ expression or action for targeting these inhibitory agents to cancer cells.

Another example of an inhibitor of C/EBPβ expression is a C/EBPβ specific short hairpin RNA (shRNA). Designing and cloning strategies for constructing shRNA expression vectors are known in the art (McIntyre and Fanning, BMC Biotechnology, 2006, 6, 1).

In still a further aspect of the present invention, activation of C/EBPβ or increasing its expression level can render normal cells resistant to side effects of cancer therapies, thereby either lowering side effects of cancer therapy or increasing the therapeutic dosage of cancer therapy. Specific cytotoxic agents whose activity is shown in the present invention to be reduced by over-expression of C/EBPβ include ALLN, MG262, lactacystin, a combination of TNFα and IFN-γ, FasL, doxorubicin, etoposide, tunicamycin and gamma radiation.

Transport of C/EBPβ expression vectors into normal cells may be enhanced by encapsulation into liposomes or by covalent coupling to highly lipophilic agents (Soutschek et al., 2004) as discussed above. Furthermore, tissue-specific promoters may be placed upstream of the open reading frame coding for C/EBPβ to ensure its expression in specific organs, thereby protecting them from chemotherapy-associated cytotoxicity. For example, a cardiac-specific promoter (Sanbe et al., 2003) will enable to overcome the dose-limiting cardiac toxicity of doxorubicin chemotherapy.

Alternatively, inducers of C/EBPβ activity and/or expression (hereinafter: C/EBPβ activators) may be employed. Said C/EBPβ activators may be rendered membrane permeable by attachment of lipophilic moieties or by their encapsulation in suitable liposomes. Ligands specific to receptors on normal cells may be conjugated to said C/EBPβ activators for targeting these conjugates to normal cells.

Thus, an inhibitor of C/EBPβ targeted to a cancer cell and/or an activator of C/EBPβ targeted to normal cell may be used in concert with cancer therapy. One, example of normal cells that may be targeted with C/EBPβ activators is hematopoietic stem cells. Hematopoietic stem cells, which are very sensitive to cancer therapy, have various specific receptors that can be used to target C/EBPβ activators. In one embodiment of the invention normal hematopoietic stem cells may be collected from the cancer patient before initiation of the cancer therapy and reinstated into the patient after introduction of the C/EBPβ activator ex-vivo.

At least one agent such as C/EBPβ or a mutein, isoform, fused protein, functional derivative, active fraction, circularly permutated derivative or a salt thereof; an agent capable of up-regulating C/EBPβ level and/or activity; and an inhibitor of a natural inhibitor of C/EBPβ level and/or activity may be used in the manufacture of a medicament for increasing resistance or lowering sensitivity of normal cells to a cancer therapy and thus preventing or treating side effects of the cancer therapy.

The term “inhibitor of a protein” within the context of this invention refers to any agent such as a protein, e.g., an antibody, polynucleotide, e.g., antisense and Small Interfering RNAs, and small molecule capable of down-regulating the production and/or action of a protein in such a way that said protein production and/or action is attenuated, reduced, or partially, substantially or completely prevented or blocked.

The term “inhibitor of C/EBPβ” within the context of this invention refers to any molecule modulating C/EBPβ production and/or action in such a way that C/EBPβ production and/or action is attenuated, reduced, or partially, substantially or completely prevented or blocked. The term “C/EBPβ inhibitor” is meant to encompass inhibitors of C/EBPβ production as well as of inhibitors of C/EBPβ action.

An inhibitor of C/EBPβ production can be any molecule negatively affecting the synthesis, processing or maturation of C/EBPβ. The inhibitors considered according to the invention can be, for example, suppressors of gene expression of the C/EBPβ, antisense mRNAs or double stranded RNA like small interfering RNA (Hunter et al., 1975) for reducing or preventing the transcription of the C/EBPβ mRNA or leading to degradation of the mRNA, proteins impairing correct folding of C/EBPβ, proteases degrading C/EBPβ, once it has been synthesized. An inhibitor of C/EBPβ action can be an antagonist of C/EBPβ. Antagonists can either bind to or sequester C/EBPβ molecule itself with sufficient affinity and specificity to partially or substantially neutralize the C/EBPβ.

Examples of inhibitors of C/EBPβ include, but are not limited to, CHOP-10 (also known as GADD153 and DDIT-3) and inducers of CHOP-10 such as tunicamycin. Several studies have correlated CHOP-10 expression with cell death, but a mechanistic link between CHOP-10 and apoptosis has never been demonstrated (McCullough et al., 2001). In one study, a role of CHOP-10 in down regulation of the survival factor Bcl2 during ER stress has been established (McCullough et al., 2001). However, a role for CHOP-10-mediated inhibition of C/EBPβ in preventing protection by the latter from a broad range of cell-death inducers as shown in the present invention has not been demonstrated so far.

Other small inhibitory molecules, which specifically inhibit C/EBPβproduction or action may be identified by proper screening of chemical or similar libraries. A small inhibitory molecule may be an organic (carbon containing) or inorganic compound with a molecular weight of about 100 to 5,000; 200 to 5,000; 200 to 2000; or 200 to 1,000 Daltons. Small molecules include, but are not limited to, metabolites, metabolic analogues, peptides, peptidomimetics, amino acids, amino acid analogs, polynucleotides, polynucleotide analogs, nucleotides, nucleotide analogs, heteroorganic and organometallic compounds. For example, small C/EBPβ inhibitory molecules may be constituted of peptides or DNA fragments including C/EBPβ recognition sites that successfully compete for C/EBPβ binding.

Inhibitors of C/EBPβ may be identified by screening libraries of chemicals or natural agents using a reporter vector assay. Briefly, a reporter vector is a mammalian expression vector consisting of a promoter responsive to C/EBPβ placed upstream of a reporter such as luciferase. Example of such reporter vector is pGL3(1272), consisting of the human IL-18BP promoter placed upstream of the luciferase coding region (Hurgin et al., 2002). In a typical screening assay mammalian cells are transfected with a mixture of vectors pGL3(1272) and a mammalian expression vector of C/EBPβ. Various chemicals and other compounds are tested for their ability to inhibit luciferase expression.

Inhibitors of C/EBPβ action may also be C/EBPβ antibodies, such as polyclonal or monoclonal antibodies, or any other agent or molecule preventing the binding of C/EBPβ to its targets, thus diminishing or preventing triggering of the reactions mediated by C/EBPβ. Antibodies or other proteins may be inserted into mammalian cells in vitro and in vivo upon mixing them with suitable reagents that are available from several manufacturers. Example of such a reagent is Chariot™, supplied by Active Motif Inc., Carlsbad, Calif. (U.S. Pat. No. 6,841,535). The term “antibody” is meant to include polyclonal antibodies, monoclonal antibodies (MAbs), chimeric antibodies, anti-idiotypic (anti-Id) antibodies to antibodies that can be labeled in soluble or bound form, and humanized antibodies as well as fragments thereof provided by any known technique, such as, but not limited to enzymatic cleavage, peptide synthesis or recombinant techniques.

A monoclonal antibody contains a substantially homogeneous population of antibodies specific to antigens, which populations contain substantially similar epitope binding sites. Mabs may be obtained by methods known to those skilled in the art. See, e.g., Kohler and Milstein, Nature, 1975, 256, 495-497; U.S. Pat. No. 4,376,110; Ausubel et al., eds., Harlow and Lane ANTIBODIES: A LABORATORY MANUAL, Cold Spring Harbor Laboratory (1988); and Colligan et al., eds., Current Protocols in Immunology, Greene Publishing Assoc. and Wiley Interscience N.Y., (1992-1996), the contents of which references are incorporated entirely herein by reference. Such antibodies may be of any immunoglobulin class including IgG, IgM, IgE, IgA, GILD and any subclass thereof. A hybridoma producing a mAb of the present invention may be cultivated in vitro, in situ or in vivo. Production of high titers of Mabs in vivo or in situ makes this the presently preferred method of production.

Chimeric antibodies are molecules of which different portions are derived from different animal species, such as those having the variable region derived from a murine Mab and a human immunoglobulin constant region. Chimeric antibodies are primarily used to reduce immunogenicity in application and to increase yields in production, for example, where murine Mabs have higher yields from hybridomas but higher immunogenicity in humans, such that human/murine chimeric Mabs are used. Chimeric antibodies and methods for their production are known in the art (Cabilly et al., Proc. Natl. Acad. Sci. USA, 1984, 81, 3273-3277; Morrison et al., Proc. Natl. Acad. Sci USA, 1984, 81, 6851-6855; Boulianne et al., Nature, 1984, 312, 643-646; Cabilly et al., European Patent Application No. 125023 (published Nov. 14, 1984); Neuberger et al., Nature, 1985, 314, 268-270; Taniguchi et al., European Patent Application No. 171496 (published Feb. 19, 1985); Morrison et al., European Patent Application No. 173494 (published Mar. 5, 1986); Neuberger et al., International Publication No. WO 8601533 (published Mar. 13, 1986); Kudo et al., European Patent Application No. 184187 (published Jun. 11, 1986); Sahagan et al., J. Immunol., 1986, 137, 1066-1074; Robinson et al., International Publication No. WO 8702671 (published May 7, 1987); Liu et al., Proc. Natl. Acad. Sci. USA, 1987, 84, 3439-3443; Sun et al., Proc. Natl. Acad. Sci. USA, 1987, 84, 214-218; Better et al. Science, 1988, 240, 1041-1043; Riechmann et al., Nature, 332:323-327; and Harlow and Lane, ANTIBODIES: A LABORATORY MANUAL, supra. These references are entirely incorporated herein by reference.

“Fully humanized antibodies” are molecules containing both the variable and constant region of the human immunoglobulin. Fully humanized antibodies can be potentially used for therapeutic use, where repeated treatments are required for chronic and relapsing diseases such as autoimmune diseases. One method for the preparation of fully human antibodies consist of “humanization” of the mouse humoral immune system, i.e. production of mouse strains able to produce human Ig (Xenomice), by the introduction of human immunoglobulin (Ig) loci into mice in which the endogenous Ig genes have been inactivated. The Ig loci are exceedingly complex in terms of both their physical structure and the gene rearrangement and expression processes required to ultimately produce a broad immune response. Antibody diversity is primarily generated by combinatorial rearrangement between different V, D, and J genes present in the Ig loci. These loci also contain the interspersed regulatory elements, which control antibody expression, allelic exclusion, class switching and affinity maturation. Introduction of unrearranged human Ig transgenes into mice has demonstrated that the mouse recombination machinery is compatible with human genes. Furthermore, hybridomas secreting antigen specific hu-mAbs of various isotypes can be obtained by Xenomice immunization with antigen.

Fully humanized antibodies and methods for their production are known in the art (Mendez et al., Nature Genetics, 1997, 15, 146-156; Buggemann et al., Eur. J. Immunol., 1991, 21, 1323-1326; Tomizuka et al., Proc. Natl. Acad. Sci. USA, 2000, 97, 722-727, International Publication No. WO 98/24893.

A monoclonal antibody is said to be “capable of binding” a molecule if it is capable of specifically reacting with the molecule to thereby bind the molecule to the antibody. The term “epitope” is meant to refer to that portion of any molecule capable of being bound by an antibody, which can also be recognized by that antibody. Epitopes or “antigenic determinants” usually consist of chemically active surface groupings of molecules such as amino acids or sugar side chains and have specific three-dimensional structural characteristics as well as specific charge characteristics.

Mutants of C/EBPβ may also be used as modulators of C/EBPβ activity. Thus, mutants exhibiting C/EBPβ activity may be used to increase resistance of non-cancerous cells to the cytotoxic effects of chemotherapy (referred herein as “muteins”). Conversly, mutants that compete and inhibit C/EBPβ activity may be used as dominant-negative agents (referred herein as “dominant-negative mutants”) thereby increasing the responsiveness of tumor cells to cancer therapy. Said muteins need to be introduced into cells by means described above for other types of proteins. As used herein the term “muteins” refers to analogs of a C/EBPβ, in which one or more of the amino acid residues of the naturally occurring components of C/EBPβ are replaced by different amino acid residues, or are deleted, or one or more amino acid residues are added to the original sequence of a C/EBPβ, without changing considerably the activity of the resulting products as compared with the original C/EBPβ. These muteins are prepared by known synthesis and/or by site-directed mutagenesis techniques, or any other known technique suitable therefore.

Muteins used in accordance with the present invention include proteins encoded by a nucleic acid, such as DNA or RNA, which hybridizes to DNA or RNA, which encodes an C/EBPβ, in accordance with the present invention, under stringent conditions. The term “stringent conditions” refers to hybridization and subsequent washing conditions, which those of ordinary skill in the art conventionally refer to as “stringent”. See Ausubel et al., Current Protocols in Molecular Biology, supra, Interscience, N.Y., §§6.3 and 6.4 (1987, 1992), and Sambrook et al. (Sambrook, J. C., Fritsch, E. F., and Maniatis, T. (1989) Molecular Cloning: A Laboratory Manual, Cold Spring Harbor Laboratory Press, Cold Spring Harbor, N.Y.).

Without limitation, examples of stringent conditions include washing conditions 12 20° C. below the calculated Tm of the hybrid under study in, e.g., 2×SSC and 0.5% SDS for 5 minutes, 2×SSC and 0.1% SDS for 15 minutes; 0.1×SSC and 0.5% SDS at 37° C. for 30 60 minutes and then, a 0.1×SSC and 0.5% SDS at 68° C. for 30 60 minutes. Those of ordinary skill in this art understand that stringency conditions also depend on the length of the DNA sequences, oligonucleotide probes (such as 10 40 bases) or mixed oligonucleotide probes. If mixed probes are used, it is preferable to use tetramethyl ammonium chloride (TMAC) instead of SSC. See Ausubel, supra.

Any such mutein preferably has a sequence of amino acids sufficiently duplicative of that of C/EBPβ, such as to have substantially similar, or even better, activity to C/EBPβ.

Characteristic activity of C/EBPβ is its transcriptional activity. Thus, it can be determined whether any given mutein has at least substantially the same activity as C/EBPβ by means of routine experimentation. For example, an assay mentioned earlier employing a reporter vector with a promoter responsive to C/EBP may be used. As long as the mutant has a similar profile of transcriptional activity it can be considered to have substantially similar activity to C/EBPβ.

In a preferred embodiment, any such mutein has at least 40% identity or homology with the sequence of C/EBPβ. More preferably, it has at least 50%, at least 60%, at least 70%, at least 80% or, most preferably, at least 90% identity or homology thereto.

Identity reflects a relationship between two or more polypeptide sequences or two or more polynucleotide sequences, determined by comparing the sequences. In general, identity refers to an exact nucleotide-to-nucleotide or amino acid-to-amino acid correspondence of the two polynucleotides or two polypeptide sequences, respectively, over the length of the sequences being compared.

For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

Methods for comparing the identity and homology of two or more sequences are well known in the art. Thus for instance, programs available in the Wisconsin Sequence Analysis Package, version 9.1 (Devereux et al., 1984), for example the programs BESTFIT and GAP, may be used to determine the % identity between two polynucleotides and the % identity and the % homology between two polypeptide sequences. BESTFIT uses the “local homology” algorithm of Smith and Waterman (1981) and finds the best single region of similarity between two sequences. Other programs for determining identity and/or similarity between sequences are also known in the art, for instance the BLAST family of programs (Altschul et al., 1990, Altschul et al., 1997, accessible through the home page of the NCBI at www.ncbi.nlm.nih.gov) and FASTA (Pearson W R, 1990; Pearson 1988).

Muteins of C/EBPβ, which can be used in accordance with the present invention, or nucleic acid coding thereof, include a finite set of substantially corresponding sequences as substitution peptides or polynucleotides which can be routinely obtained by one of ordinary skill in the art, without undue experimentation, based on the teachings and guidance presented herein.

Preferred changes for muteins in accordance with the present invention are what are known as “conservative” substitutions. Conservative amino acid substitutions of C/EBPβ may include synonymous amino acids within a group, which have sufficiently similar physicochemical properties that substitution between members of the group will preserve the biological function of the molecule (Grantham, 1974). It is clear that insertions and deletions of amino acids may also be made in the above-defined sequences without altering their function, particularly if the insertions or deletions only involve a few amino acids, e.g., under thirty, and preferably under ten, and do not remove or displace amino acids which are critical to a functional conformation, e.g., cysteine residues. Proteins and muteins produced by such deletions and/or insertions come within the purview of the present invention.

The synonymous amino acid groups are preferably those defined in Table A, more preferably those defined in Table B, most preferably those defined in Table C.

TABLE A
Preferred Groups of Synonymous Amino Acids

| Amino Acid | Synonymous Group |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Ala, Thr, Pro, Ser, Gly |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Ser, Thr, Cys |
| His | Glu, Lys, Gln, Thr, Arg, His |
| Gln | Glu, Lys, Asn, His, Thr, Arg, Gln |
| Asn | Gln, Asp, Ser, Asn |
| Lys | Glu, Gln, His, Arg, Lys |
| Asp | Glu, Asn, Asp |
| Glu | Asp, Lys, Asn, Gln, His, Arg, Glu |
| Met | Phe, Ile, Val, Leu, Met |
| Trp | Trp |

TABLE B
More Preferred Groups of Synonymous Amino Acids

| Amino Acid | Synonymous Group |
|---|---|
| Ser | Ser |
| Arg | His, Lys, Arg |
| Leu | Leu, Ile, Phe, Met |
| Pro | Ala, Pro |
| Thr | Thr |
| Ala | Pro, Ala |
| Val | Val, Met, Ile |
| Gly | Gly |
| Ile | Ile, Met, Phe, Val, Leu |
| Phe | Met, Tyr, Ile, Leu, Phe |
| Tyr | Phe, Tyr |
| Cys | Cys, Ser |
| His | His, Gln, Arg |
| Gln | Glu, Gln, His |
| Asn | Asp, Asn |
| Lys | Lys, Arg |
| Asp | Asp, Asn |
| Glu | Glu, Gln |
| Met | Met, Phe, Ile, Val, Leu |
| Trp | Trp |

TABLE C
Most Preferred Groups of Synonymous Amino Acids

| Amino Acid | Synonymous Group |
|---|---|
| Ser | Ser |
| Arg | Arg |
| Leu | Leu, Ile, Met |
| Pro | Pro |
| Thr | Thr |
| Ala | Ala |
| Val | Val |
| Gly | Gly |
| Ile | Ile, Met, Leu |
| Phe | Phe |
| Tyr | Tyr |
| Cys | Cys, Ser |
| His | His |
| Gln | Gln |
| Asn | Asn |
| Lys | Lys |
| Asp | Asp |
| Glu | Glu |
| Met | Met, Ile, Leu |
| Trp | Met |

Examples of production of amino acid substitutions in proteins which can be used for obtaining muteins of C/EBPβ polypeptides, for use in the present invention include any known method steps, such as presented in U.S. Pat. Nos. 4,959,314, 4,588,585 and 4,737,462, to Mark et al.; 5,116,943 to Koths et al.; 4,965,195 to Namen et al.; 4,879,111 to Chong et al.; and 5,017,691 to Lee et al.; and lysine substituted proteins presented in U.S. Pat. No. 4,904,584 (Shaw et al.).

The term “fused protein” refers to a polypeptide comprising a C/EBPβ, or a mutein or fragment thereof, fused with another protein, which, e.g., has an extended residence time in body fluids. A C/EBPβ may thus be fused to, e.g., an immunoglobulin or a fragment thereof.

“Functional derivatives” as used herein cover derivatives of C/EBPβ, and their muteins and fused proteins, which may be prepared from the functional groups which occur as side chains on the residues or the N or C terminal groups, by means known in the art, and are included in the invention as long as they remain pharmaceutically acceptable, i.e. they do not destroy the activity of the protein which is substantially similar to the activity of C/EBPβ.

These derivatives may, for example, include polyethylene glycol side chains, which may mask antigenic sites and extend the residence of a C/EBPβ in body fluids. Other derivatives include aliphatic esters of the carboxyl groups, amides of the carboxyl groups by reaction with ammonia or with primary or secondary amines, N acyl derivatives of free amino groups of the amino acid residues formed with acyl moieties (e.g. alkanoyl or carbocyclic aroyl groups) or O acyl derivatives of free hydroxyl groups (for example that of seryl or threonyl residues) formed with acyl moieties.

An “active fraction” according to the present invention may be, e.g., a fragment of C/EBPβ. The term fragment refers to any subset of the molecule, that is, a shorter peptide that retains the desired biological activity. Fragments may be readily prepared by removing amino acids from either end of the C/EBPβ molecule and testing the resultant fragment for transcriptional activity. Proteases for removing one amino acid at a time from either the N-terminal or the C-terminal of a polypeptide are known, and so determining fragments, which retain the desired biological activity, involves only routine experimentation.

As active fractions of an C/EBPβ, muteins and fused proteins thereof, the present invention further covers any fragment or precursors of the polypeptide chain of the protein molecule alone or together with associated molecules or residues linked thereto, e.g., sugar or phosphate residues, or aggregates of the protein molecule or the sugar residues by themselves, provided said fraction has substantially similar activity to C/EBPβ.

The term “salts” herein refers to both salts of carboxyl groups and to acid addition salts of amino groups of the C/EBPβ molecule or analogs thereof. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids, such as, e.g., hydrochloric acid or sulfuric acid, and salts with organic acids, such as, for example, acetic acid or oxalic acid. Of course, any such salts must retain the biological activity of C/EBPβ.

“Isoforms” of C/EBPβ are proteins capable of transcriptional activity or fragment thereof, which may be produced by alternative splicing or alternative translation start-site.

The term “circularly permuted derivatives” as used herein refers to a linear molecule in which the termini have been joined together, either directly or through a linker, to produce a circular molecule, and then the circular molecule is opened at another location to produce a new linear molecule with termini different from the termini in the original molecule. Circular permutations include those molecules whose structure is equivalent to a molecule that has been circularized and then opened. Thus, a circularly permuted molecule may be synthesized de novo as a linear molecule and never go through a circularization and opening step. The preparation of circularly permutated derivatives is described in WO95/27732.

Some substances according to the invention such as peptides, proteins, oligonucleotides and/or small molecules necessitate their introduction into cells of a living organism. For this purpose, it is desired to improve membrane permeability of peptides, proteins, oligonucleotides and/or small molecules. Derivatization with lipophilic structures may be used in creating peptides and proteins with enhanced membrane permeability. For instance, the sequence of a known membranotropic peptide as noted above may be added to the sequence of the peptides, proteins and oligonucleotides according to the invention. Further, peptides and proteins, may be derivatized by partly lipophilic structures such as the above-noted hydrocarbon chains, which are substituted with at least one polar or charged group. For example, lauroyl derivatives of peptides have been described by Muranishi et al. (1991). Further modifications of peptides and proteins comprise the oxidation of methionine residues to thereby create sulfoxide groups, as described by Zacharia et al. (1991). Zacharia and co-workers also describe peptide or derivatives wherein the relatively hydrophobic peptide bond is replaced by its ketomethylene isoester (COCH₂). These and other modifications known to the person of skill in the art of protein and peptide chemistry enhance membrane permeability.

Another way of enhancing membrane permeability is the use receptors, such as virus receptors, on cell surfaces in order to induce cellular uptake peptides, proteins, oligonucleotides and/or small molecules. This mechanism is used frequently by viruses, which bind specifically to certain cell surface molecules. Upon binding, the cell takes the virus up into its interior. The cell surface molecule is called a virus receptor. For instance, the integrin molecules CAR and AdV have been described as virus receptors for Adenovirus, see Hemmi et al. (1998) and references therein. The CD4, GPR1, GPR15, and STRL33 molecules have been identified as receptors/co-receptors for HIV, see Edinger et al. (1998) and references therein.

Thus, conjugating peptides, proteins, oligonucleotides and/or small molecules to molecules that are known to bind to cell surface receptors will enhance membrane permeability of said peptides, proteins, oligonucleotides and/or small molecules. Examples for suitable groups for forming conjugates are sugars, vitamins, hormones, cytokines, transferrin, asialoglycoprotein, and the like molecules. Low et al., U.S. Pat. No. 5,108,921, describes the use of these molecules for the purpose of enhancing membrane permeability of peptides, proteins and oligonucleotides, and the preparation of said conjugates. Low and co-workers further show that molecules such as folate or biotin may be used to target the conjugate to a multitude of cells in an organism, because of the abundant and unspecific expression of the receptors for these molecules.

The above use of cell surface proteins for enhancing membrane permeability of peptides, proteins, oligonucleotides and/or small molecules may also be used in targeting said peptides, proteins, oligonucleotides and/or small molecules to certain cell types or tissues. For instance, if it is desired to target cancer cells, it is preferable to use a cell surface protein that is expressed more abundantly on the surface of those cells. Examples are the folate receptor, the mucin antigens MUC1, MUC2, MUC3, MUC4, MUC5AC, MUC5B, and MUC7, the glycoprotein antigens KSA, carcinoembryonic antigen, prostate-specific membrane antigen (PSMA), HER-2/neu, and human chorionic gonadotropin-beta. Wang et al. (1998) teaches the use of folate to target cancer cells, and Zhang et al. (1998) teaches the relative abundance of each of the other antigens noted above in various types of cancer and in normal cells.

The term “agent capable of up-regulating a protein” or “activator of a protein” within the context of this invention refers to any agent or activator, such as a protein, nucleotide, polynucleotide and small molecule, capable of up-regulating said protein production and/or action.

Activators of C/EBPβ may be identified by screening libraries of chemicals or natural agents using a reporter vector assay. Briefly, a reporter vector is a mammalian expression vector consisting of a promoter responsive to C/EBPβ placed upstream of a reporter such as luciferase. Example of such reporter vector is pGL3(1272), consisting of the human IL-18BP promoter placed upstream of the luciferase coding region (Hurgin et al., 2002). In a typical screening assay mammalian cells are transfected with vector pGL3(1272) with or without a mammalian expression vector of IRF-1. Various chemicals and other compounds are tested for their ability to replace the C/EBPβ expression vector as enhancers of luciferase expression as described (Hurgin et al., 2002).

The peptides, proteins, oligonucleotides and/or small molecules may therefore, using the above-described conjugation techniques, be targeted to certain cell type as desired. For instance, if it is desired to target cells of the lymphocytic lineage, a peptides, proteins, oligonucleotides and/or small molecules may be targeted at such cells, for instance, by using the MHC class II molecules that are expressed on these cells. This may be achieved by coupling an antibody, or the antigen-binding site thereof, directed against the constant region of said MHC class II molecule to peptides, proteins, oligonucleotides and/or small molecules. Further, numerous cell surface receptors for various cytokines and other cell communication molecules have been described, and many of these molecules are expressed with in more or less tissue- or cell-type restricted fashion. Thus, when it is desired to target a subgroup of T cells, the CD4 T cell surface molecule may be used for producing the conjugate with peptides, proteins, oligonucleotides and/or small molecules. CD4-binding molecules are provided by the HIV virus, whose surface antigen gp42 is capable of specifically binding to the CD4 molecule. Peptides, proteins, oligonucleotides and/or small molecules may be advantageously targeted to T cells.

Peptides, proteins, oligonucleotides and/or small molecules may be introduced into cells by the use of a viral vector. The use of vaccinia vector for this purpose is detailed in chapter 16 of Current Protocols in Molecular Biology. The use of adenovirus vectors has been described e.g. by Teoh et al (1998), Narumi et al. (1998), Pederson et al. (1998), Guang-Lin et al. (1998) and references therein, Nishida et al. (1998), Schwarzenberger et al. (1998), and Cao et al. (1998). Retroviral transfer of antisense sequences has been described by Daniel et al. (1998).

When using viruses as vectors, the viral surface proteins are generally used to target the virus. As many viruses, such as the above adenovirus, are rather unspecific in their cellular tropism, it may be desirable to impart further specificity by using a cell-type or tissue-specific promoter. Griscelli et al., 1998 teach the use of the ventricle-specific cardiac myosin light chain 2 promoter for heart-specific targeting of a gene whose transfer is mediated by adenovirus.

Alternatively, the viral vector may be engineered to express an additional protein on its surface, or the surface protein of the viral vector may be changed to incorporate a desired peptide sequence. The viral vector may thus be engineered to express one or more additional epitopes, which may be used to target, said viral vector. For instance, cytokine epitopes, MHC class II-binding peptides, or epitopes derived from homing molecules may be used to target the viral vector in accordance with the teaching of the invention.

An expression vector comprises a promoter, optionally an intron sequence and splicing donor/acceptor signals, and further optionally comprises a termination sequence. Expression vectors are well known in the art and are described in Current Protocols in Molecular Biology, for example in chapter 16.

The use of a vector for inducing and/or enhancing the endogenous production of C/EBPβ, is also contemplated according to the invention. The vector may comprise regulatory sequences capable of enhancing the expression of C/EBPβ. Such regulatory sequences may be, for example, promoters or enhancers. The regulatory sequence may then be introduced into the right locus of the genome by homologous recombination, thus operably linking the regulatory sequence with the gene, the expression of which is required to be induced or enhanced. The technology is usually referred to as “endogenous gene activation” (EGA), and it is described, e.g., in WO 91/09955.

The present invention provides also pharmaceutical compositions including peptides, proteins, oligonucleotides and/or small molecules according to the present invention and a pharmaceutically acceptable carrier. For example, pharmaceutical compositions may comprise for increasing resistance of normal cells to cancer therapy at least one of the following agents: (i) C/EBPβ or a mutein, isoform, fused protein, functional derivative, active fraction, circularly permutated derivative or a salt thereof; (ii) an agent capable of up-regulating C/EBPβ level and/or activity; and (iii) an inhibitor of C/EBPβ level and/or activity, and a pharmaceutically acceptable carrier, as described above. For decreasing the resistance or increasing sensitivity of cancer cells to cancer therapy, pharmaceutical compositions may comprise an inhibitor of C/EBPβ level and/or activity and a pharmaceutically acceptable carrier.

The pharmaceutical composition of the present invention includes a sufficient amount of substance(s) according to the invention to achieve its intended purpose. In addition, the pharmaceutical compositions may contain suitable pharmaceutically acceptable carriers comprising excipients and auxiliaries, which facilitate processing of the active compounds into preparations which can be used pharmaceutically and which can stabilize such preparations for administration to the patient in need thereof as well known to those of skill in the art.

The substances according to the invention might be administered to a patient in need thereof in a variety of ways. The routes of administration include intraliver, intradermal, transdermal (e.g. in slow release formulations), intramuscular, intraperitoneal, intravenous, subcutaneous, oral, epidural, topical, and intranasal routes. In addition the substance can be administered together with other components of biologically active agents such as pharmaceutically acceptable surfactants, excipients, carriers, diluents and vehicles.

The dosage administered, as single or multiple doses, to an individual will vary depending upon a variety of factors, including the substance pharmacokinetic properties, the route of administration, patient conditions and characteristics (sex, age, body weight, health, size), extent of symptoms, concurrent treatments, frequency of treatment and the effect desired. Adjustment and manipulation of established dosage ranges are well within the ability of those skilled.

The definition of “pharmaceutically acceptable” is meant to encompass any carrier, which does not interfere with effectiveness of the biological activity of the active ingredient and that is not toxic to the host to which it is administered. For example, for parenteral administration, the substance according to the invention may be formulated in a unit dosage form for injection in vehicles such as saline, dextrose solution, serum albumin and Ringer's solution.

A “therapeutically effective amount” is such that when administered, the said substances of the invention induce a beneficial effect in cancer therapy and/or in preventing or treating side effects of cancer therapy. The dosage administered, as single or multiple doses, to an individual may vary depending upon a variety of factors, including the route of administration, patient conditions and characteristics (sex, age, body weight, health, size), extent of symptoms, concurrent treatments, frequency of treatment and the effect desired. Adjustment and manipulation of established dosage ranges are well within the ability of those skilled in the art.

The compounds with which the invention is concerned may be prepared for administration by any route consistent with their pharmacokinetic properties.

The active ingredient may also be administered parenterally in a sterile medium. Depending on the vehicle and concentration used, the drug can either be suspended or dissolved in the vehicle.

The term “dosage” relates to the determination and regulation of the frequency and number of doses.

All references cited herein, including journal articles or abstracts, published or unpublished U.S. or foreign patent application, issued U.S. or foreign patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not any way an admission that any aspect, description or embodiment of the present invention is disclosed, taught or suggested in the relevant art.

Having now described the invention, it will be more readily understood by reference to the following examples that are provided by way of illustration and are not intended to be limiting of the present invention.

### EXAMPLES

### Example 1

### Over-Expression of C/EBPβ Protects Cells from Death Induced by the Non-Specific Proteasome Inhibitor ALLN

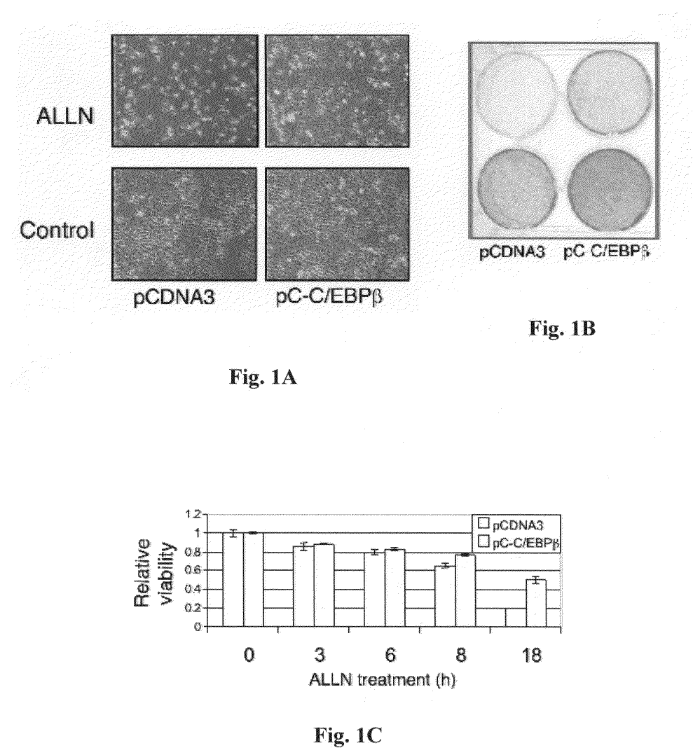

Primary tumor cells and cell lines express relatively high levels of the transcription factor C/EBPβ. We studied the effect of C/EBPβ expression level on cell survival following challenge by cell death inducers. For this purpose, human WISH cells (2×10⁵/ml in MEM and 10% FBS) were seeded in 6 well plates (2 ml/well), incubated for 20 h, and transfected with either pC-C/EBPβ expression vector or empty vector pC-DNA3 (1 μg/well total) using JetPI (Polyplus transfection Inc.) as a transfection aid. 24 h after transfection, the medium was replaced with fresh medium and ALLN in DMF was added to the medium at a concentration of 26 μM. 24 h after ALLN addition the cultures were observed under the microscope (FIG. 1A) and then stained with crystal violet (FIG. 1B). Cell density was determined at different times following addition of ALLN by the trypan blue dye exclusion method. Relative viability was determined by dividing with the number of cells at time 0 (FIG. 1C). No difference was observed between control cells transfected with C/EBPβ expression vector (pC-C/EBPβ) and cells transfected with empty vector (pC-DNA3) at times 0-8 h. After 18 hours exposure to ALLN a significant higher survival was observed in cells transfected with the C/EBPβ expression vector as compared to cells transfected with the empty vector (FIG. 1C).

### Example 2

### Over-Expression of C/EBPβ Protects Cells from Death Induced by Different Agents

To evaluate the generality of the C/EBPβ-mediated protection from cell death inducers, we repeated the experiment of Example 1 testing a broad range of cell-death inducers, including Fas ligand (FasL), doxorubicin, the specific proteasome inhibitors lactacystin and MG262, which are known to induce apoptosis.

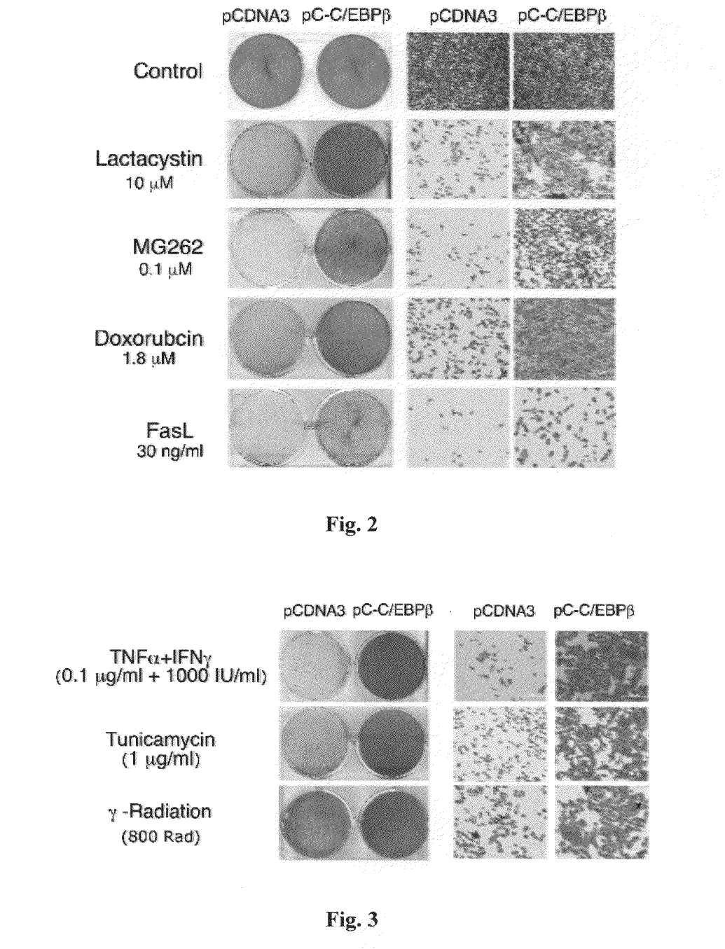

For this purpose, human WISH cells were seeded in 6-well plates, transfected with either pC-C/EBPβ expression vector or pCDNA3 as described in the previous Example. At 24 h after transfection the medium was replaced with fresh medium and lactacystin (10 μM, Sigma), MG262 (0.1 μM, Sigma), doxorubicin (1.8 μM, Sigma) or FasL (30 ng/ml, Peprotech) was added to the medium. 24 h after addition of the cell death agent to the medium, the cultures were stained with crystal violet (FIG. 2, left) and observed under the microscope (FIG. 2, right).

No difference was observed in cells, which were not exposed to agents inducing cell death and which were transfected with the C/EBPβ expression vector or with the empty vector. A significant reduction in cell death was observed in cells exposed to each one of the tested death inducer agents transfected with the C/EBPβ expression vector as compared with exposed cells transfected with the empty vector.

We repeated the experiments exploring the effect of over expression of C/EBPβ with additional death inducers such as TNFα plus IFNγ, tunicamycin or gamma radiation. For this purpose, human WISH cells were seeded in 6-well plates, transfected with either pC-C/EBPβ expression vector or pCDNA3 as described in Example 1. At 24 h after transfection the medium was replaced with fresh medium and TNFα (100 ng/ml, Peprotech) plus IFNγ (1000 IU/ml, Peprotech) or tunicamycin (1 μg/ml, Sigma) were added to the medium. Alternatively, after the transfection the cultures were subjected to gamma radiation (800 Rad). After 24 h. the cultures were stained with crystal violet and observed macroscopically (FIG. 3, left) and microscopically (FIG. 3, right). A significant reduction in cell-death with each one of the tested death inducer agents, including gamma radiation was observed in cells transfected with the C/EBPβ expression vector as compared with cells transfected with the empty vector.

The results obtained indicate that human cells over-expressing C/EBPβ are more resistant to inducers of cell death.

### Example 3

### Over-Expression of C/EBPβ Protects Cells from Inducers of Cell Apoptosis

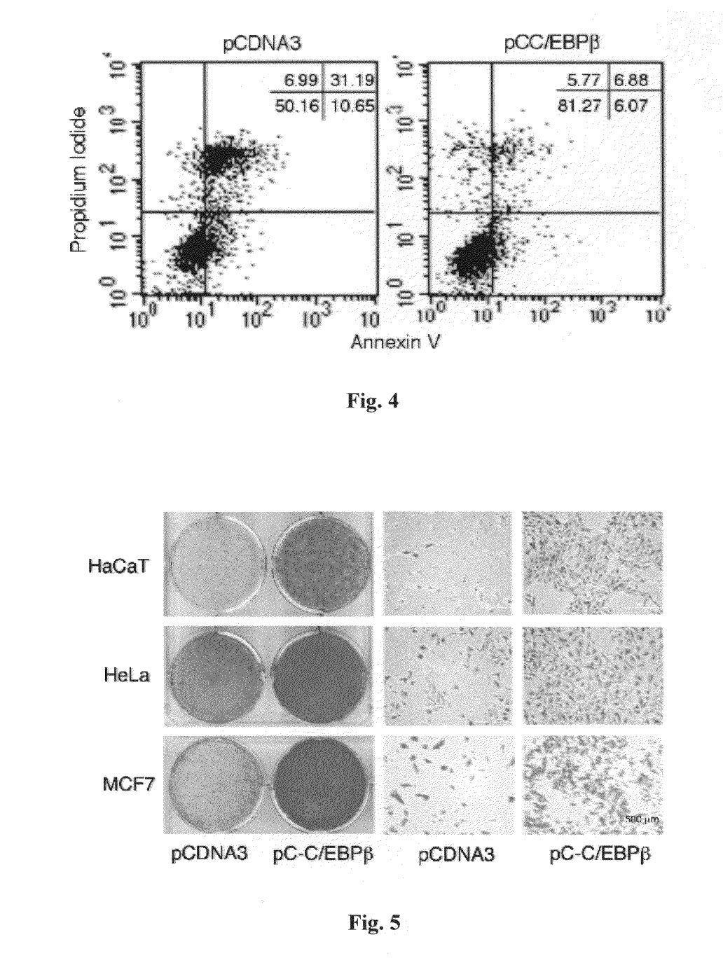

The following experiment was carried out to discriminate between the possible effects of C/EBPβ on cell proliferation and on apoptosis. For this purpose, human WISH cells were seeded and transfected with either pC-C/EBPβ expression vector or pCDNA3 as described in Example 1. At 24 h after transfection the medium was replaced with fresh medium and the proteasome inhibitor MG262 (0.1 μM, final) was added. After 12 h the cultures were collected by trypsin digestion, stained with propidium iodide (Sigma) and antibody to Annexin V (Bender MedSystems, Vienna) and analyzed by a cell sorter. A significant inhibition of both early and late apoptosis by C/EBPβ over-expression was seen (upper and lower right squares, indicating that C/EBPβ over-expression affects apoptosis rather than the rate of cell proliferation (FIG. 4).

### Example 4

### Over-Expression of C/EBPβ Protects Different Types of Cells from Inducers of Cell Death

We tested the effect of over-expression of C/EBPβ on survival of different cell lines exposed to different cell death inducers. Thus, human HaCat keratinocytes, HeLa (cervical carcinoma) or MCF7 cells (breast carcinoma) were seeded (2×10⁵/ml in MEM and 10% FBS) in 6 well plates (2 ml/well). After 20 h the cultures were transfected with either pC-C/EBPβ expression vector or pCDNA3 (1 μg/well total) using JetPI (Polyplus transfection Inc.) as a transfection aid. After 24 h the medium was replaced and either TNFα (100 ng/ml) plus IFNγ (1000 IU/ml) in HaCaT and HeLa cells or doxorubicin (1.8 μM) in MCF7 cells were added. After 24 h the cultures were stained with crystal violet and observed macroscopically (FIG. 5, left) and microscopically (FIG. 5, right). It was observed that over expression of C/EBPβ protected HaCaT keratinocytes and HeLa cells from cell death induced by the combination of TNFα and IFNγ, and MCF7 cells from doxorubicin.

The results show significant protection from apoptosis by different cell death inducing agents in different types of cells over-expressing C/EBPβ.

### Example 5

### Inhibition of C/EBPβ-Induced Resistance to Cell Death by Over-Expression of the C/EBPβ Antagonist Chop-10

In order to demonstrate that the protective effect observed following transfection with C/EBPβ expression vector is indeed mediated by over-expression of C/EBPβ we explored the effect of CHOP-10 (also known as Gadd-153), a known inhibitor of C/EBPβ activity.

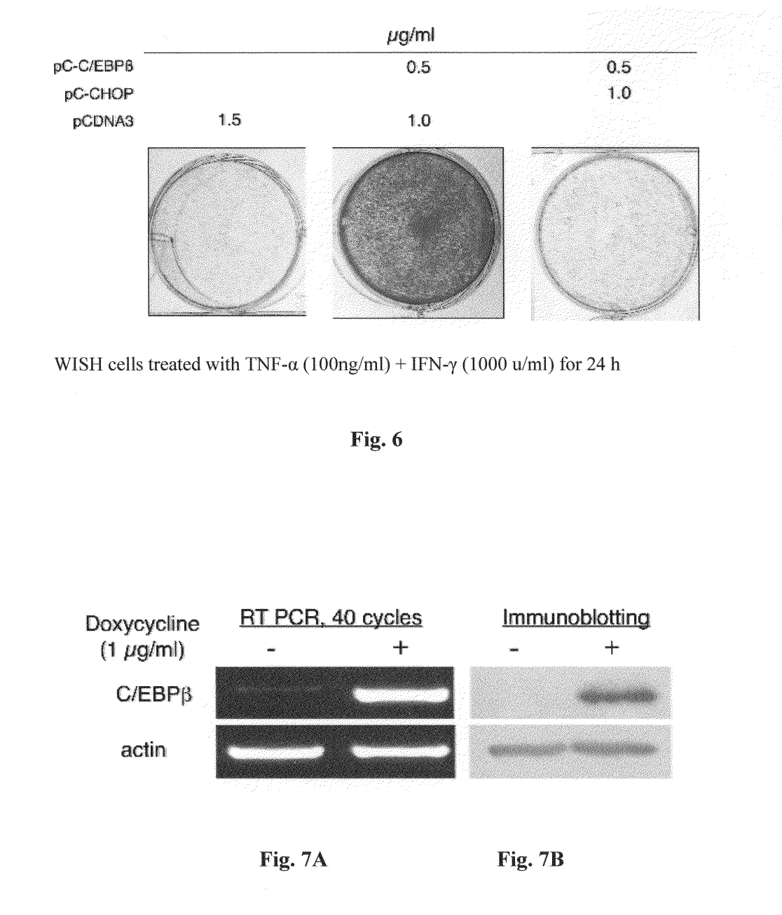

For this purpose human WISH cells (2×10⁵/ml in MEM and 10% FBS) were seeded in 6 well plates (2 ml/well). After 20 h the cultures were transfected with pCDNA3 alone (1.5 μg/ml), pC-C/EBPβ expression vector plus pCDNA3 (0.5 and 1.0 μg/ml, respectively) or pC-C/EBPβ plus pC-CHOP (0.5 and 1.0 μg/ml, respectively), using JetPI (Polyplus transfection Inc.) as a transfection aid. After 24 h the medium was replaced and TNFα (100 ng/ml) plus IFNγ (1000 IU/ml) were added. After 24 h the cultures were stained with crystal violet and observed macroscopically (FIG. 6). A significant protection from cell death induced by TNF plus IFN-γ was observed in cells transfected with the C/EBPβ expression vector as compared with empty vector. However, co-expression of pC-C/EBPβ with pC-CHOP abolished the protective effect of pC-C/EBPβ on cell death.

### Example 6

### B16 Melanoma Cells Stably Transfected and Expressing C/EBPβ from an Inducible C/EBPβ Vector are Protected from Inducers of Cell Death

The following experiment was carried out in order to confirm that the protective effect of C/EBPβ from cell death inducers is independent of the transfection process. Thus, B16 mouse melanoma cells, stably transfected with an inducible C/EBPβ expression vector (Clone F-10.9) were obtained from M. Revel and J. Chebath, Weizmann Institute of Science, Israel. Induction of C/EBPβ expression was triggered by addition of the antibiotic doxycycline (1 μg/ml, 24 h, Sigma) to the culture medium and was evaluated by both RT-PCR and immunoblotting. RT-PCR was performed as follows: F-10.9 cells (10⁶) were harvested and total RNA was extracted by using the Rneasy® kit- from QIAGEN. cDNA was prepared by using random hexamers (Invitrogen) and M-MLV Reverse Transcriptase (Promega) according to the manufacturer's instructions. PCR was performed with the following primers: mouse C/EBPβ, 5′-GAGCTGACGGCGGAGAACGA and 5′-ACCCCGCAGGAACATCTTTA. Amplifications were done by initial denaturation (95° C., 4 min), 27 cycles of denaturation (95° C., 80 s), annealing (57° C., 60 s) and extension (72° C., 80 s), and final extension (72° C., 5 min). The resulting PCR products were resolved by agarose (1%) gel electrophoresis. A significant induction of C/EBPβ mRNA was seen upon treatment with doxycycline (FIG. 7A). For immunoblotting, cells were washed three times with ice-cold PBS and immediately frozen in liquid nitrogen. Cell pellets were re-suspended in four packed cell volumes of cytoplasmic buffer (10 mM Hepes, pH 7.9, 10 mM NaCl, 0.2 mM EDTA, 5% glycerol, 1.5 mM MgCl₂, 1 mM DTT, 0.5 mM PMSF, 50 mM NaF, 0.1 mM Na₃VO₄, 2 mM EGTA, 10 mM Na₂MoO₄, 2 μg/ml each of leupeptin, pepstatin, and aprotinin). The suspension was freeze/thawn twice, incubated on ice for 20 min and then centrifuged (3,000×g, 10 min, 4° C.). The supernatant containing the proteins was collected and protein concentration was determined by a BCA Protein assay kit (Pierce), using BSA as a standard. Protein extracts (10 μg) were boiled in SDS/PAGE sample buffer containing 25 mM DTT and the supernatant was resolved by SDS/PAGE (10% acrylamide). The gel was then blotted onto a nitrocellulose membrane and proteins were detected with the indicated antibodies. Immune complexes were identified with a Super Signal detection kit (Pierce.). A significant induction of C/EBPβ was seen upon treatment with doxycycline (FIG. 7B).

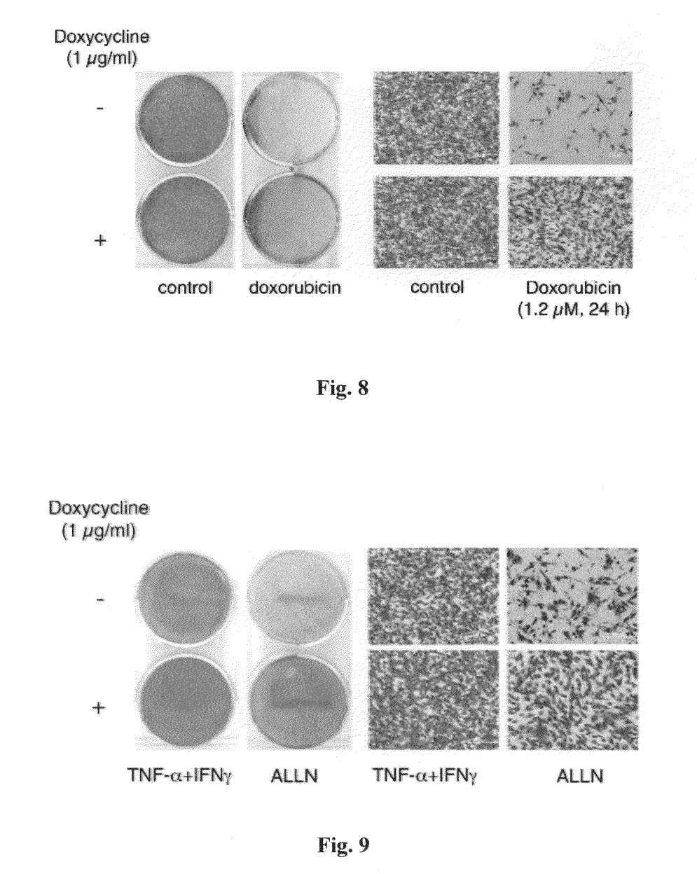

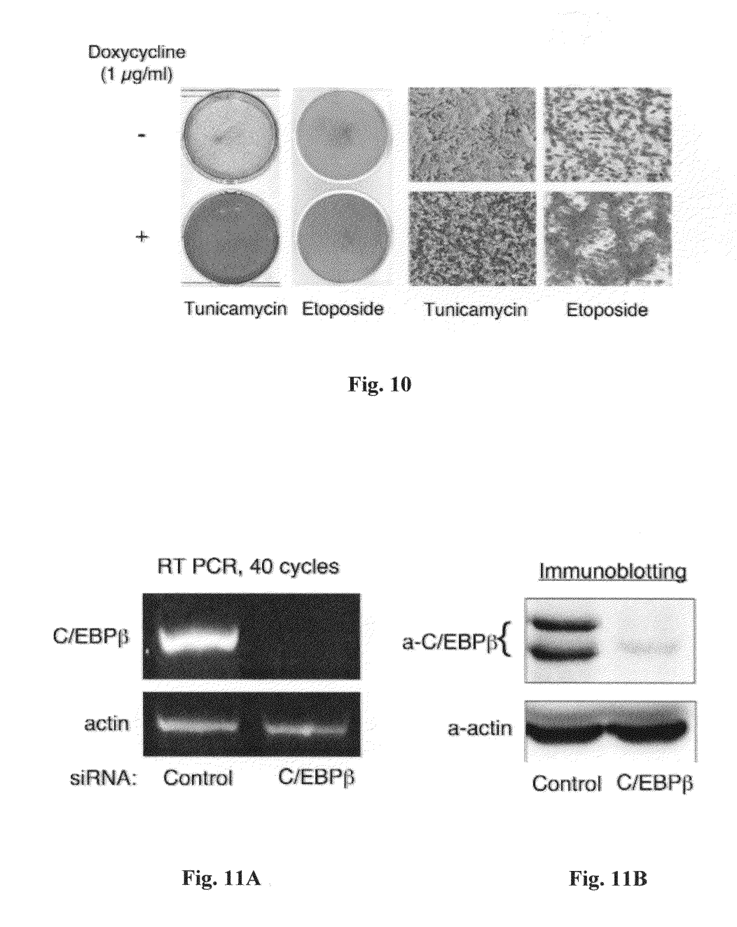

B16 Clone F-10.9 cells (2×10⁵/ml in MEM and 10% FBS) were then seeded in 6 well plates (2 ml/well). After 24 h doxycycline (1 μg/ml) was added and after 12 h doxorubicin (1.2 μM) was added. After 24 h the cultures were stained with crystal violet and observed macroscopically (FIG. 8, left) and microscopically (FIG. 8, right). No difference was noted between cells that were not exposed to the death inducer and which were induced to produce C/EBPβ or were not induced. Enhanced survival was noted in cells that were exposed to the death inducer and which were induced to produce C/EBPβ as compared to non-induced cells. Thus, significant protection from apoptosis was observed in cells induced to express C/EBPβ as compared with non-induced cells (FIG. 8). Similar results were seen with other inducers of cell death, including TNFα (100 ng/ml) plus IFN-γ (1000 IU/ml) added to the cells for 72 h and ALLN (26 μM) added to the cells for 48 h. (FIG. 9) as well as tunicamycin (0.5 μg/ml) added to the cells for 24 h and etoposide (100 μM) added to the cells for 72 h (FIG. 10).

### Example 7

### Knockdown of Endogenous C/EBPβ in Cells Renders these Cells Highly Sensitive to Killing by Death Inducers

We checked the effect of silencing C/EBPβ with C/EBPβ specific siRNA in order to determine the role of endogenously expressed C/EBPβ in cell death induced by the proteasome inhibitor MG262.

For this purpose, human WISH cells (2×10⁵/ml in MEM and 10% FBS, without antibiotics) were seeded in 6 well plates (2 ml/well). After 20 h the cultures were transfected with control or C/EBPβ siRNA pools purchased from Dharmacon (4×25 nM/well in Dharmafect 1; 48 h; Dharmacon). The siRNA pools comprised the following sequences: 5′CCAAGAAGACCGUGGACAA, 5′GCAAGAAGCCGGCCGAGUA, 5′AAUCCAUGGAAGUGGCCAA and 5′CCGCGGACUGCAAGCGGAARNA. RNA and protein were then analyzed by RT-PCR as in Example 6, using the following primers: human C/EBPβ, 5′-GAAGTGGCCAACTTCTACTAC and 5′—CGCCTGGTAGCCGAGGTAAG. Immunoblotting was performed as described in Example 6 with rabbit antibodies directed against C/EBPβ (a-C/EBPβ; Santa Cruz). A significant inhibition of C/EBPβ expression was demonstrated both at the mRNA (FIG. 11A) and protein (FIG. 11B) levels.

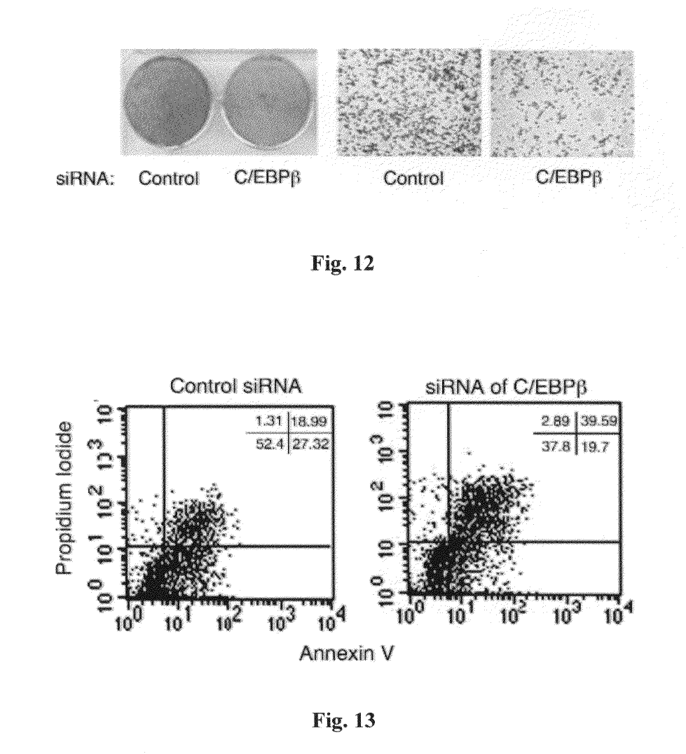

Next, human WISH cells (2×10⁵/ml in MEM and 10% FBS, without antibiotics) were seeded in 6 well plates (2 ml/well). After 20 h the cultures were transfected with control or C/EBPβ siRNA pools (4×25 nM/well in Dharmafect 1; 48 h; Dharmacon). The medium was replaced and the cells were then treated with MG262 (0.1 μM). After 24 h the cultures were stained with crystal violet and observed macroscopically (FIG. 12, left) and microscopically (FIG. 12, right). The results summarized in FIG. 12 show a significant increase in cell death in cultures transfected with the C/EBPβ siRNA as compared with cultures transfected with control siRNA.

These results show that reduction of basal C/EBPβ increases the sensitivity of cells to an inducer of cell death.

### Example 8

### Analysis of the Effect of C/EBPβ Ablation on Induction of Apoptosis of WISH Cells by the Proteasome Inhibitor MG262

Human WISH cells (2×10⁵/ml in MEM and 10% FBS, without antibiotics) were seeded in 6 well plates (2 ml/well). After 20 h the cultures were transfected with control or C/EBPβ siRNA pools (4×25 nM/well in Dharmafect 1; 48 h; Dharmacon). The medium was replaced and the cells were then treated with MG262 (0.1 μM). After 8 h the cells were collected by trypsin digestion, stained with propidium iodide and antibody to Annexin V and analyzed by a cell sorter. A significant increase of late apoptosis was seen in cells transfected with the C/EBPβ siRNA as compared with control siRNA, indicating that inhibition of endogenous C/EBPβ by siRNA increases their apoptotic response to MG262 (FIG. 13).

These results show that ablation of basal C/EBPβ increases the sensitivity or lowers the resistance of cells to killing by inducers of apoptosis.

### Example 9

### Analysis of the Effect of C/EBPβ Ablation on Induction of Apoptosis of WISH Cells by the Cell Death Inducers ALLN or TNFα Plus IFNγ

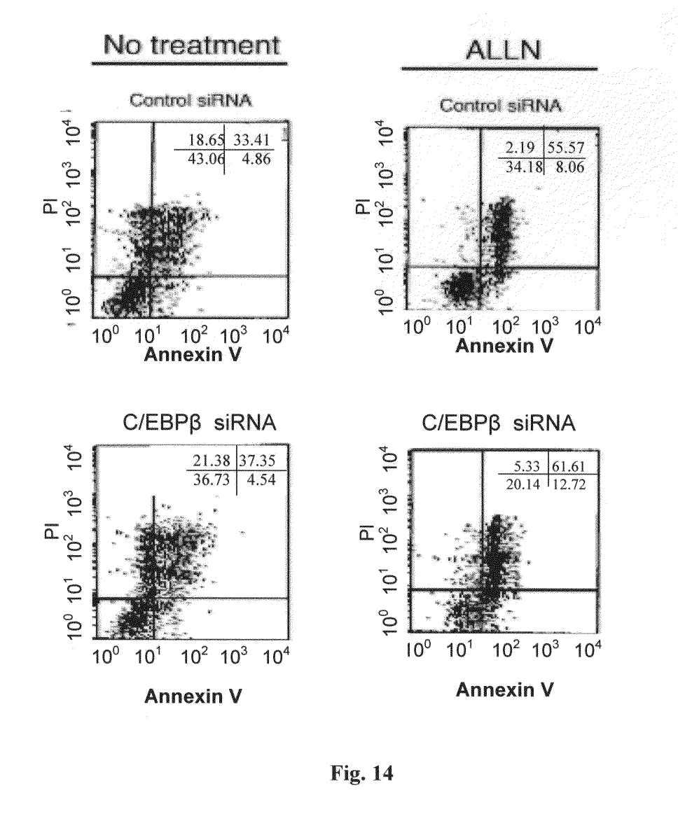

To evaluate the generality of the concept that reducing C/EBPβ enhances the sensitivity of cells to inducers of cell death, we repeated the experiment of Example 8 testing more cell-death inducers. Human WISH cells (2×10⁵/ml in MEM and 10% FBS, without antibiotics) were seeded in 6 well plates (2 ml/well). After 20 h the cultures were transfected with control siRNA or C/EBPβ siRNA pools (4×25 nM/well in Dharmafect 1; 48 h; Dharmacon). The medium was replaced and the cells were then either treated with medium alone (No treatment) or treated with ALLN (26 μM). After 24 h the cultures were stained with anti annexin V (X axis) and propidium iodide (Y axis) and subjected to 2D fluorescence analysis cell sorting (FACS). As seen in FIG. 14, after exposure to the death inducer a significant increase in cell death, due to late apoptosis (compare upper right corners), was observed in cultures transfected with the C/EBPβ siRNA as compared with cultures transfected with control siRNA.

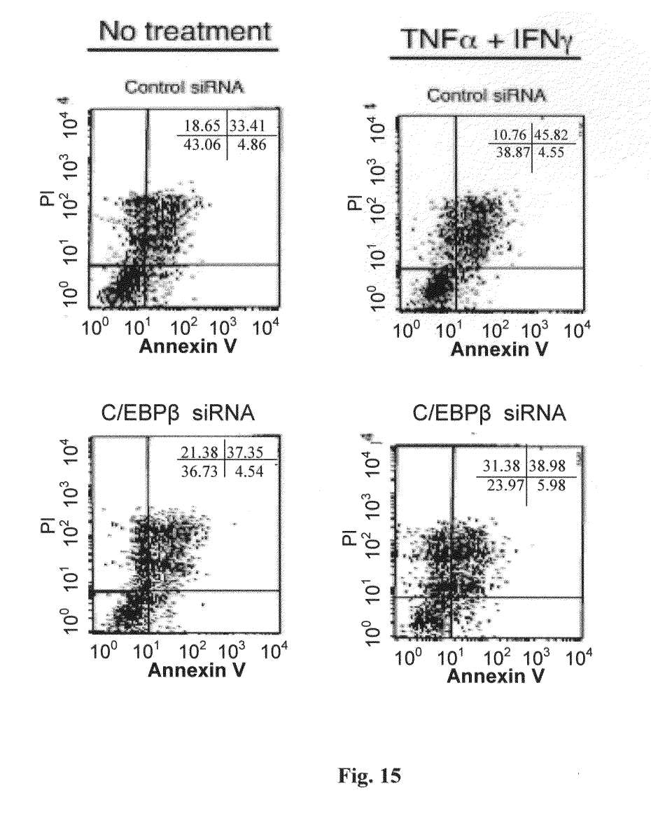

Human WISH cells (2×10⁵/ml in MEM and 10% FBS, without antibiotics) were seeded in 6 well plates (2 ml/well). After 20 h the cultures were transfected with control siRNA or C/EBPβ siRNA pools (4×25 nM/well in Dharmafect 1; 48 h; Dharmacon). The medium was replaced and the cells were then either treated with medium alone (No treatment) or treated with TNFα (100 ng/ml) plus IFNγ (1000 IU/ml). After 24 h the cultures were stained with anti annexin V (X axis) and propidium iodide (Y axis) and subjected to 2D fluorescence analysis cell sorting (FACS). As seen in FIG. 15, after exposure to the death inducer a significant increase in cell death, due to late apoptosis (compare upper right corners), occurred in cultures transfected with the C/EBPβ siRNA as compared with cultures transfected with control siRNA.

In all, these results show that reducing of basal C/EBPβ by siRNA increased the sensitivity of cells to different inducers of apoptotic cell death.

### Example 10

### Analysis of the Effect of C/EBPβ Ablation on Induction of Apoptosis of Human HeLa Cell and Human MCF7 Cells by TNFα Plus IFNγ

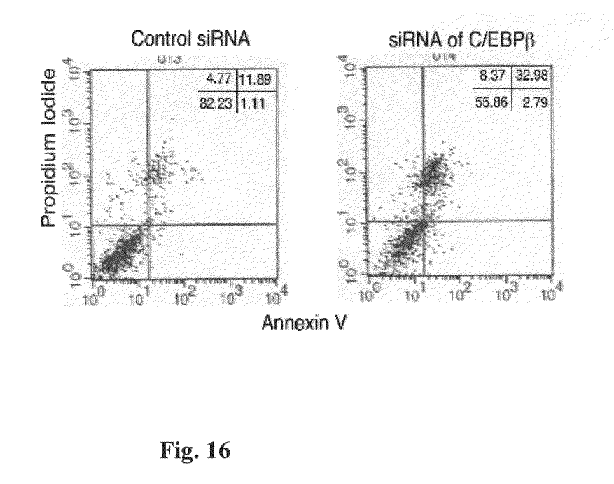

We tested the effect of knockout of C/EBPβ on induction of cell death by cell death agent on cell lines derived from tumors. For this purpose, human HeLa (cervical cancer) cells (2×10⁵/ml in MEM and 10% FBS, without antibiotics) were seeded in 6 well plates (2 ml/well). After 20 h the cultures were transfected with control or C/EBPβ siRNA pools (4×25 nM/well in Dharmafect 1; 48 h; Dharmacon). The medium was replaced and the cells were then treated with TNFα (100 ng/ml) plus IFNγ (1000 IU/ml). After 24 h the cultures were stained with crystal violet and observed macroscopically and microscopically. A significant increase in cell death was seen in cultures transfected with the C/EBPβ siRNA as compared with cultures transfected with control siRNA (data not shown). Identical cultures were subjected to flow cytometry for estimating the extent of apoptosis using annexin and propidium iodide staining. It was found that siRNA of C/EBPβ increased the extent of late apoptosis from 11.89% to 32.98% (compare upper right squares in FIG. 16).

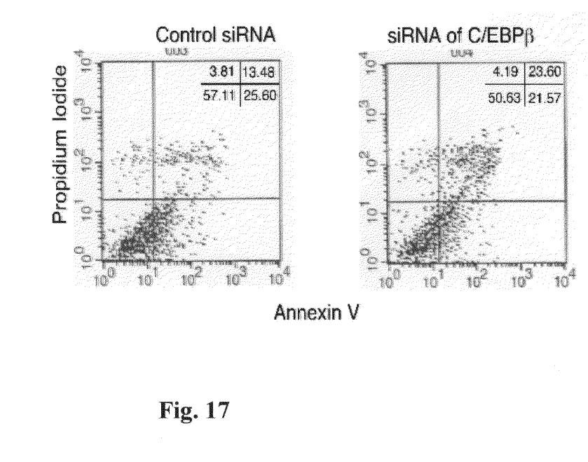

Human MCF7 (ductal breast carcinoma cells) cells (2×10⁵/ml in MEM and 10% FBS, without antibiotics) were seeded in 6 well plates (2 ml/well). After 20 h the cultures were transfected with control or C/EBPβ siRNA pools (4×25 nM/well in Dharmafect 1; 48 h; Dharmacon). The medium was replaced and the cells were then treated with TNFα (100 ng/ml) plus IFNγ (1000 IU/ml). After 24 h the cultures were stained with crystal violet and observed macroscopically and microscopically. A significant increase in cell death was seen in cultures transfected with the C/EBPβ siRNA as compared with cultures transfected with control siRNA (data not shown). Identical cultures were subjected to flow cytometry for estimating the extent of apoptosis using annexin and propidium iodide staining. It was found that siRNA of C/EBPβ increased the extent of late apoptosis from 13.48% to 23.60% (compare upper right squares in FIG. 17).

### Example 11

### Protection from Inducers of Cell Death by C/EBPβ is not Mediated by p53

It was recently reported that C/EBPβ binds to p53 and inhibits its activity, part of which could be induction of apoptosis. We carried out several experiments to find out if the protective effect of C/EBPβ over-expression against apoptosis induced by cell death inducers involves inhibition of p53. Initially, we determined which of the cell death inducers used for induction of apoptosis elevates the level of cellular p53. For this purpose, WISH cells were treated with; etoposide (50 μM for 24 h); MG262 (0.1 μM, for 24 h); TNFα (100 ng/ml) plus IFN-γ (1000 IU/ml) for 48 h; doxorubicin (1.8 μM, for 24 h); vincristine (0.1 μg/ml, for 24 h); FasL (60 ng/ml, 24 h); tunicamycin (1 μg/ml, for 24 h) or ALLN (26 μM, for 24 h), and extracts of these cells were subjected to immunoblotting with anti p53 (mab dd1+1801, 1:40 from M. Oren, WIS) as described in Example 6.

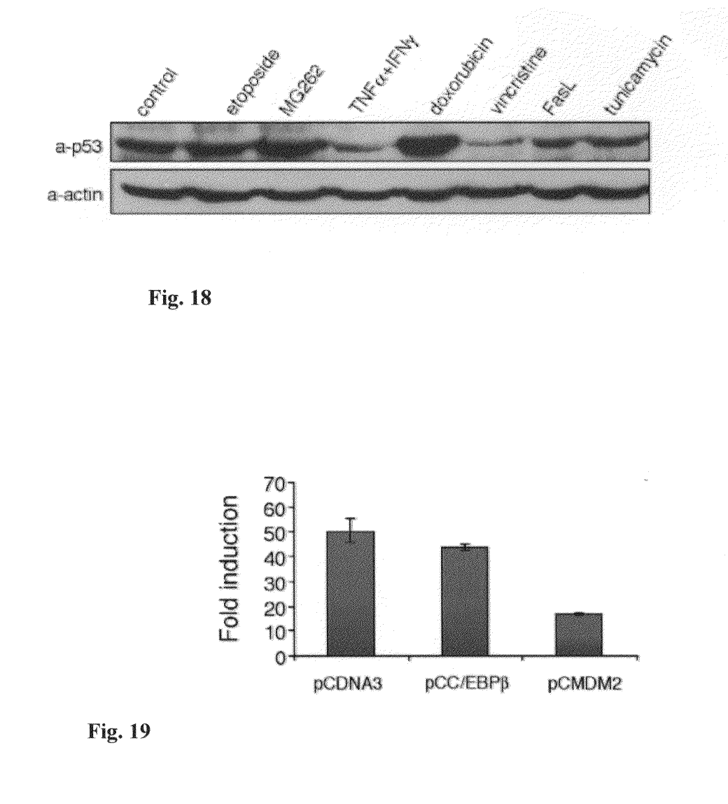

We found that etoposide, TNF plus IFN-γ, vincristine, FasL and tunicamycin did not induce p53 in human WISH cells as determined by immunoblotting with antibody directed against p53, whereas extensive induction of p53 was observed with MG262 and doxorubicin (FIG. 18).

Next, WISH cells were transfected with either pC-C/EBPβ, pCMDM2 (a positive control for p53 inhibition) or control vector (0.5 μg/ml, 2 ml), together with a p53 reporter vector pRGC-Luc (Osada et al., 1998), 0.25 μg/ml) and renilla pRL-sv40 (15 ng/ml). Luciferase activity of p53 was then determined. Following induction of p53 by doxorubicin, we found that pCMDM2 effectively inhibited p53 activity whereas only a slight inhibition was seen with pC-C/EBPβ (FIG. 19).

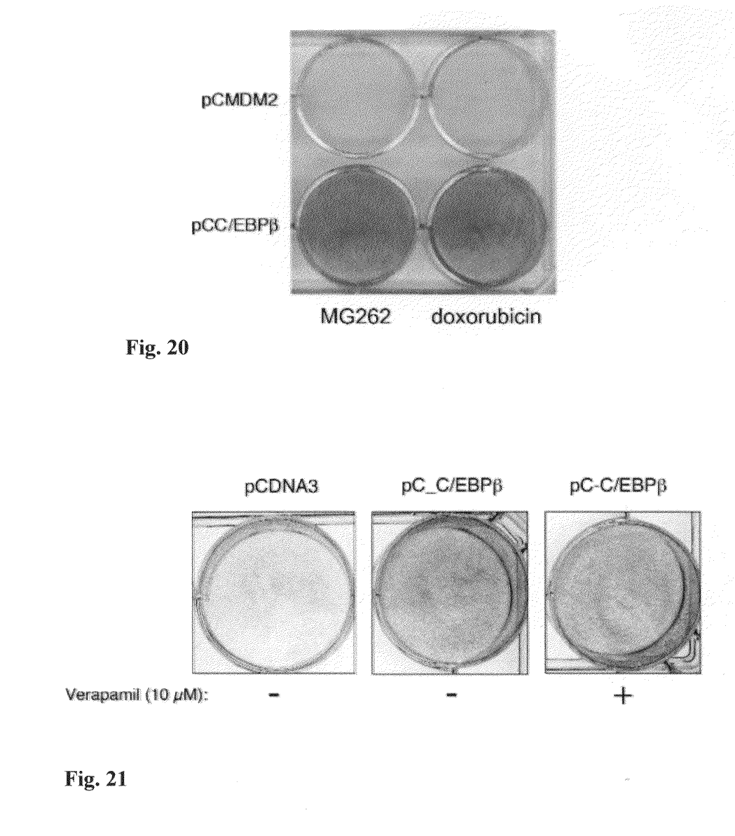

We then tested if MDM2 could mimic C/EBPβ in protecting from apoptosis triggered by p53 inducers. For this purpose, WISH cells were transfected with either pCC/EBPβ or pCMDM2 (0.5 mg/ml, 2 ml) and treated the cells with MG262 (0.1 μM) or doxorubicin (1.8 μM), both for 24 h. It was observed that both MG262 and doxorubicin induced massive apoptosis in cells transfected with pC-MDM2 whereas cells transfected with pC-C/EBPβ were protected (FIG. 20).

These results show that C/EBPβ-mediated protection from inducers of cell apoptosis is not mediated by inhibition of p53.

### Example 12

### Protection from Inducers of Cell Death by C/EBPβ is not Mediated by Known C/EBPβ-Induced Genes

A C/EBPβ response element was identified in promoters of several genes encoding pro-survival factors such as multidrug resistance gene 1(MDR1), IGF-1, IL-6, and AKT. We carried out experiments to find out whether MDR1, IGF-1, IL-6, or AKT are mediators of the C/EBPβ-induced protective effect.

MDR1 is a pump that removes various chemotherapeutic agents. It is inhibited by verapamil. WISH cells were transfected with the C/EBPβ vector or empty vector (0.5 μg/ml, 2 ml) and then treated MG262 (0.1 μM) plus verapamil (10 μM) for 24 h. We found that verapamil did not reverse the C/EPP-β-induced protective effect (FIG. 21). Thus, the protective effect of C/EBPβ is not mediated by induction of MDR1.

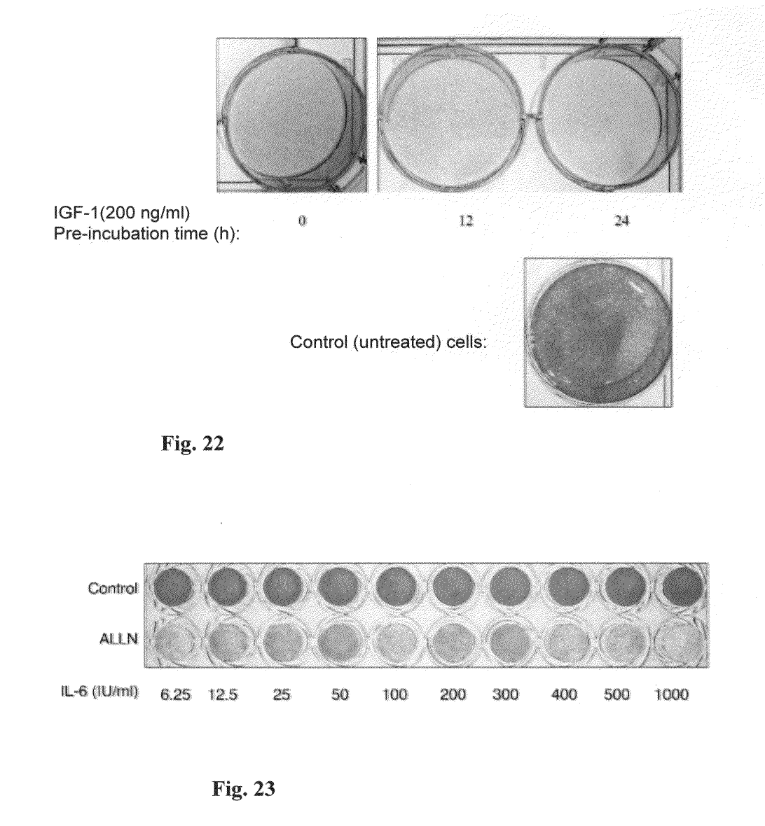

WISH cells were pre-incubated with IGF-1 (200 ng/ml) for the indicated times. Doxorubicin (2 μM, for 16 h) was then added. Addition of IGF-1 to the culture medium did not protect WISH cells from doxorubicin (FIG. 22). The same results were obtained when apoptosis was induced by MG262, TNF-α plus IFN-γ, and tunicamycin (data not shown).

WISH Cells (30,000 cells/well) were pre incubated with 6.25, 12.5, 25, 50, 100, 200, 300, 400, 500, or 1000 IU/ml of IL-6 for 6 h and then 26 μM ALLN was added for 48 h. The results obtained show that IL-6 added 6 h before challenge did not protect cells from apoptosis induced by ALLN (FIG. 23).

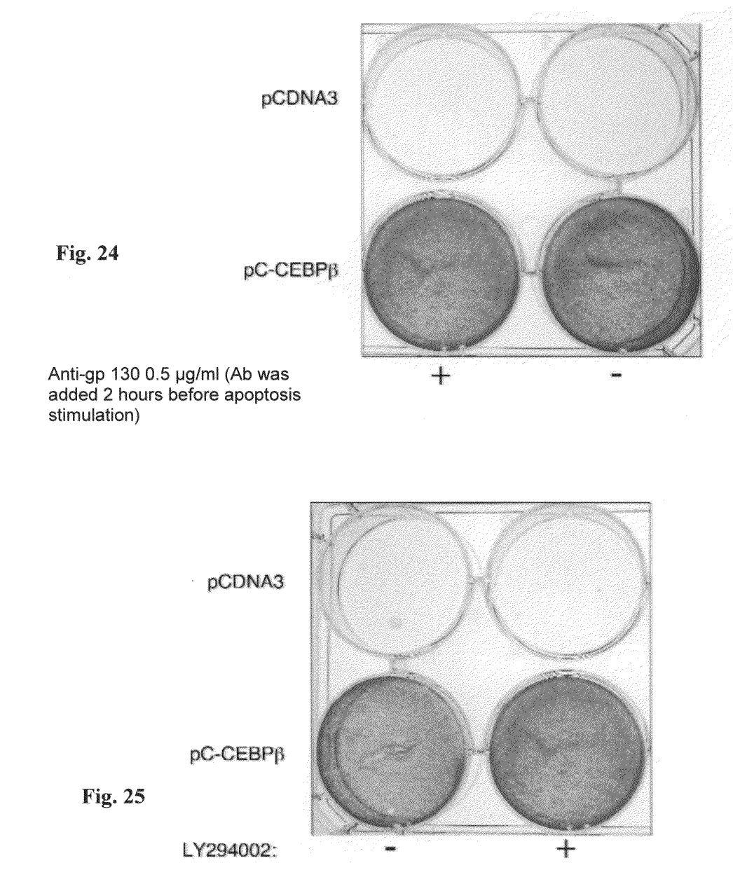

Next, WISH cells were transfected with the C/EBPβ vector or empty vector (0.5 μg/ml, 2 ml). After 24 h the cells were incubated with anti-gp130, (0.5 μg/ml, 2 h) and then treated with TNF-α (100 ng/ml)+IFN-γ (1000 IU/ml) for 22 h. The results obtained show that antibodies to the IL-6 receptor signaling sub-unit gp130 added 2 h before challenge by the combination of TNF-α and IFN-γ did not block the protective effect elicited by C/EBPβ (FIG. 24).

Akt is a serine/threonine protein kinase that has been implicated in mediating a variety of biological responses including inhibiting apoptosis and stimulating cellular growth. (Thakkar et al., 2001). Once activated, Akt exerts antiapoptotic effects through phosphorylation of substrates such as Bad (Datta et al., 1997), caspase 9 (Cardone et al., 1998), etc. which directly regulates the apoptotic machinery. The Akt inhibitor LY294002 (40) was added 2 hours before apoptosis stimulation by TNF-α (100 ng/ml)+IFN-γ (1000 IU/ml) for 22 h in WISH cells transfected either with C/EBPβ vector or with empty vector (0.5 μg/ml each, 2 ml). The results obtained show that LY294002 did not reverse the protective effect of C/EBPβ (FIG. 25).

We therefore concluded that the protective effect induced by over-expression of C/EBPβ involves a still unknown and hence not an obvious mechanism.

### Example 13

### The Role of C/EBPβ as a Modulator of Tumor Growth In Vivo

C/EBPβ is a transcription factor acting as a master regulator of cellular proliferation and differentiation, metabolism, inflammation, immune responses and numerous other responses. It is expressed as an active form termed C/EBPβ LAP (LAP) and as a shorter inactive form termed C/EBPβ LIP (LIP), using alternative translation start sites (Ramji and Foka, 2002). Importantly, LIP acts as a specific inhibitor of LAP (Descombes and Schibler, 1991). LAP is over-expressed in many types of tumors and was implicated in tumor cell growth and survival (Sebastian and Johnson, 2006; Lamb et al., 2003). A survey of primary cancer cells and cell lines identified a highly statistically significant link between cyclin D1 and C/EBPβexpression in many human cancers. Thus, together with cyclin D1, LAP accelerates cell proliferation (Lamb et al., 2003). Many other studies also implicated C/EBPβ in regulating cell death; however, a clear mechanism has not been established (Sterneck et al., 2006; Yoon et al., 2007).

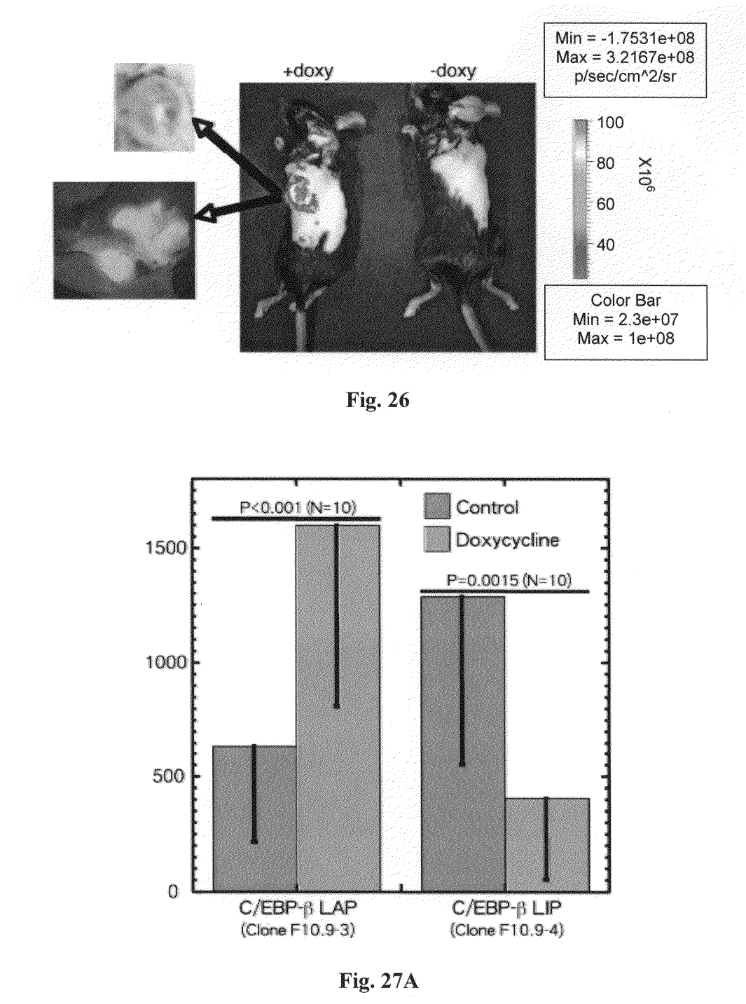

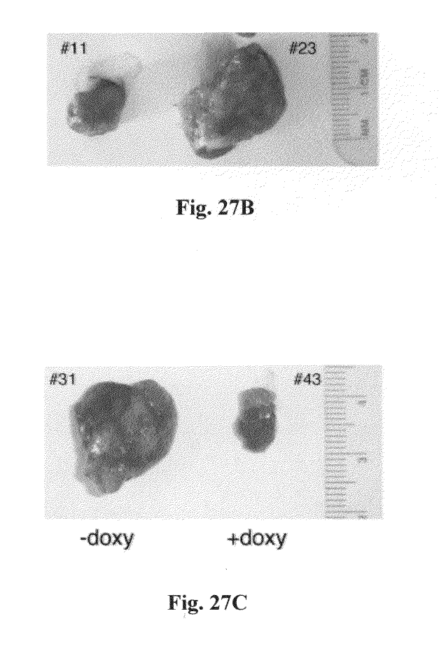

In order to test the role of C/EBPβ in tumor growth in mice, two different murine B16 melanoma clones, stably transfected with doxycycline-inducible LAP or LIP were used. First, B16 Clone F10.9-3 cells (0.5×10⁶, 100 μl) together with matrigel (100 μl, BD) were subcutaneously injected to C57B1/6 mice. Doxycycline (1 mg/ml) was added to the drinking water of the doxycycline group for 7 days until tumors were developed. FIG. 26 shows images of control and doxycycline treated tumors, shot by the iVIS digital camcorder. Then, B16 Clone F10.9-3 LAP cells or F10.9-4 LIP cells (0.5×10⁶, 100 μl each) together with matrigel (100 μl, BD) were subcutaneously injected to 4 groups of 10 C57B1/6 mice. Doxycycline (1 mg/ml) was added to the drinking water of the doxycycline groups for 13 days. The mice were then sacrificed, and the tumors were excised, weighted, photographed and preserved in formaldehyde solution. FIG. 27A shows a plot of the tumor weights (mg) in each one of the groups and FIGS. 27B-27C show photographs of typical tumors. These results show that over-expression of LAP has a remarkable tumor growth-promoting effect, after 13 days; whereas over-expression of LIP inhibit tumor growth, probably by inhibiting basal expression of endogenous LAP.

As revealed from previous studies we have conducted, LAP attenuated endoplasmic reticulum (ER) stress-induced cell death, whereas LIP augmented ER stress-induced cell death. Similarly to the in vivo studies, LAP did not affect cell proliferation or the extent of apoptosis, as determined by staining for annexin, a marker of apoptosis, and propidium iodide, a marker of membrane damage that serves to evaluate the progression of the apoptosis, followed by flow cytometry. These findings suggest that similar survival mechanisms occur in these cells both in vivo and in vitro.

### Example 14

### A Screening Assay for Modulators of C/EBPβ Activity

Human WISH cells (2×10⁵/ml in MEM and 10% FBS) were seeded in 96 well plates (0.2 ml/well), incubated for 20 h, and transfected with pC-C/EBPβ expression vector, renilla luciferase control vector (0.05 μg/well each) and reporter vector pGL3(1272), consisting of the human IL-18BP promoter placed upstream of the firefly luciferase coding region (Hurgin et al., 2002) (0.05 μg/well). Transfection was aided with JetPI (Polyplus transfection Inc.). 24 h after transfection, the medium was replaced with fresh medium containing a material to be tested at different concentrations. After 24 h luciferase activities are measured and the ratio (R) of firefly to renilla luciferase activity is determined. Inhibitors of C/EBPβ activity will be detected by observing a reduced R as compared to control wells lacking any material to be tested. Activators of C/EBPβ activity will be detected by observing a high value of R as compared to control wells lacking any material to be tested.

### REFERENCES

Buck, M. et al., (2001). “Nuclear export of phosphorylated C/EBPbeta mediates the inhibition of albumin expression by TNF-alpha.” Embo J 20(23): 6712-23
Cardone, M. H. et al., (1998). “Regulation of cell death protease caspase-9 by phosphorylation.” Science 282(5392): 1318-21
Choy, L. and R. Derynck (2003). “Transforming growth factor-beta inhibits adipocyte differentiation by Smad3 interacting with CCAAT/enhancer-binding protein (C/EBP) and repressing C/EBP transactivation function.” J Biol Chem 278(11): 9609-19
Cortes-Canteli, M. et al., (2002). “CCAAT/enhancer-binding protein beta plays a regulatory role in differentiation and apoptosis of neuroblastoma cells.” J Biol Chem 277(7): 5460-7
Datta, S. R. et al., (1997). “Akt phosphorylation of BAD couples survival signals to the cell-intrinsic death machinery.” Cell 91(2): 231-41
Descombes, P. and U. Schibler (1991). “A liver-enriched transcriptional activator protein, LAP, and a transcriptional inhibitory protein, LIP, are translated from the same mRNA.” Cell 67(3): 569-79
Dorsett, Y. and T. Tuschl (2004). “siRNAs: applications in functional genomics and potential as therapeutics.” Nat Rev Drug Discov 3(4): 318-29
Homma, J. et al., (2006). “Increased expression of CCAAT/enhancer binding protein beta correlates with prognosis in glioma patients.” Oncol Rep 15(3): 595-601
Hurgin, V. et al., (2002). “The promoter of IL-18 binding protein: activation by an IFN-gamma-induced complex of IFN regulatory factor 1 and CCAAT/enhancer binding protein beta.” Proc Natl Acad Sci USA 99(26): 16957-62
Kim, D. H., M. A. Behlke, et al. (2005). “Synthetic dsRNA Dicer substrates enhance RNAi potency and efficacy.” Nat Biotechnol 23(2): 222-6
Lamb, J. et al. (2003). “A mechanism of cyclin D1 action encoded in the patterns of gene expression in human cancer.” Cell 114(3): 323-34
Li, W. et al., (2005). “A gene expression signature for relapse of primary wilms tumors.” Cancer Res 65(7): 2592-601
Marshall, J. et al., (2003). “Calcium channel and NMDA receptor activities differentially regulate nuclear C/EBPbeta levels to control neuronal survival.” Neuron 39(4): 625-39
McCullough, K. D. et al., (2001). “Gadd153 sensitizes cells to endoplasmic reticulum stress by down-regulating Bc12 and perturbing the cellular redox state.” Mol Cell Biol 21(4): 1249-59
Osada, M. et al., (1998). “Cloning and functional analysis of human p51, which structurally and functionally resembles p53.” Nat Med 4(7): 839-43
Ramji, D. P. and P. Foka (2002). “CCAAT/enhancer-binding proteins: structure, function and regulation.” Biochem J 365(Pt 3): 561-75
Sanbe, A. et al., (2003). “Reengineering inducible cardiac-specific transgenesis with an attenuated myosin heavy chain promoter.” Circ Res 92(6): 609-16
Sankpal, N. V. et al., (2005). “Transcriptional repression of TFF1 in gastric epithelial cells by CCAAT/enhancer binding protein-beta.” Biochim Biophys Acta 1728(1-2): 1-10
Schneider-Merck, T. et al., (2006). “Physical interaction and mutual transrepression between CCAAT/enhancer-binding protein beta and the p53 tumor suppressor.” J Biol Chem 281(1): 269-78
Sebastian, T. and P. F. Johnson (2006). “Stop and go: anti-proliferative and mitogenic functions of the transcription factor C/EBPbeta.” Cell Cycle 5(9): 953-7
Soutschek, J. et al., (2004). “Therapeutic silencing of an endogenous gene by systemic administration of modified siRNAs.” Nature 432(7014): 173-8
Sterneck, E. et al., (2006). “Conditional ablation of C/EBP beta demonstrates its keratinocytespecific requirement for cell survival and mouse skin tumorigenesis.” Oncogene 25(8): 1272-6
Thakkar, H. et al., (2001). “Pro-survival function of Akt/protein kinase B in prostate cancer cells. Relationship with TRAIL resistance.” J Biol Chem 276(42): 38361-9
Vegesna, V. et al., (2002). “C/EBP-beta, C/EBP-delta, PU.1, AML1 genes: mutational analysis in 381 samples of hematopoietic and solid malignancies.” Leuk Res 26(5): 451-7
Wessells, J. et al., (2004). “Critical prosurvival roles for C/EBP beta and insulin-like growth factor I in macrophage tumor cells.” Mol Cell Biol 24(8): 3238-50
Yoon, K. et al., (2007). “Decreased survival of C/EBP beta-deficient keratinocytes is due to aberrant regulation of p53 levels and function.” Oncogene 26(3): 360-7

1. A C/EBPβ specific siRNA selected from the group of C/EBPβspecific siRNAs consisting of the C/EBPβ specific siRNAs of SEQ ID NOs: 1-4 and chemically modified derivatives thereof.

2. The C/EBPβ specific siRNA of claim 1, wherein said chemically modified C/EBPβ specific siRNA is chol-C/EBPβ specific siRNA.

3. A pharmaceutical composition comprising a C/EBPβ specific siRNA according to claim 1, and a pharmaceutically acceptable carrier.

4. The pharmaceutical composition of claim 3, for decreasing resistance or enhancing sensitivity of cancer cells to a cancer therapy.

5. The pharmaceutical composition of claim 4, wherein said resistance of the cancer cells to said cancer therapy is not associated to p53 multidrug resistance gene 1 (MDR1), IGF-1, IL-6 or AKT activity and/or level.

6. The pharmaceutical composition of claim 4, wherein said cancer therapy induces apoptosis in cells or is selected from the group consisting of chemotherapy, cytokine therapy, proteasome inhibitor therapy, radiation therapy, or a combination thereof.

7. The pharmaceutical composition of claim 6, wherein said cytokine therapy comprises administration of FasL, IFN-γ, TNF-α or a combination thereof, and said proteasome inhibitor therapy comprises administration of MG262, lactacystin, ALLN or a combination thereof.

8. A method for decreasing resistance or enhancing sensitivity of cancer cells to a cancer therapy in an individual in need, comprising administering to said individual an effective amount of a C/EBPβ inhibitor selected from (i) a C/EBPβ specific siRNA or shRNA; or (ii) CHOP-10, a CHOP-10 expression vector and/or an inducer of CHOP-10.

9. The method of claim 8, wherein said C/EBPβ inhibitor is a C/EBPβ specific siRNA.

10. The method of claim 9, wherein said C/EBPβ specific siRNA is selected from the C/EBPβ specific siRNAs of SEQ ID NOs: 1-4.

11. The method of claim 9, wherein said C/EBPβ specific siRNA is chemically modified to increase its penetration into cancer cells.

12. The method of claim 11, wherein said chemically modified C/EBPβ specific siRNA is chol-C/EBPβ specific siRNA.

13. The method of claim 8, wherein said C/EBPβ inhibitor is a CHOP-10 expression vector.

14. The method of claim 8, wherein said C/EBPβ inhibitor is linked to a ligand that binds specifically to cancer cells.

15. The method of claim 8, wherein the resistance of the cancer cells to said cancer therapy is not associated to p53 multidrug resistance gene 1 (MDR1), IGF-1, IL-6 or AKT activity and/or level.

16. The method of claim 8, wherein said cancer therapy induces apoptosis in cells, or said cancer therapy is selected from chemotherapy, cytokine therapy, proteasome inhibitor therapy, radiation therapy or a combination thereof.

17. The method of claim 16, wherein said cytokine therapy comprises administration of FasL, IFN-γ, TNF-α or a combination thereof, and said proteasome inhibitor therapy comprises administration of MG262, lactacystin, ALLN or a combination thereof.
